(19) United States
(12) Patent Application Publication
(10) Pub. No.: US 2010/0234419 A1
(43) Pub. Date: Sep. 16, 2010

(54) SUBSTITUTED 3-AMINOISOXAZOLOPYRIDINES AS KCNQ2/3 MODULATORS

(75) Inventors: Sven KÜHNERT, Duren (DE); Gregor Bahrenberg, Monschau-Konzen (DE); Wolfgang Schröder, Aachen (DE)

Correspondence Address: Briscoe, Kurt G.;Norris McLaughlin & Marcus, PA, 875 Third Avenue, 8th Floor, New York, NY, 10022, US

(73) Assignee: GRUNENTHAL GMBH, Aachen (DE)

(21) Appl. No.: 12/720,803

(22) Filed: Mar. 10, 2010

Related U.S. Application Data
(60) Provisional application No. 61/158,803, filed on Mar. 10, 2009.

(30) Foreign Application Priority Data
Mar. 10, 2009 (EP) 09003430.7

(51) Int. Cl. A61K 31/437; C07D 498/04; A61P 29/00; A61P 25/08; A61P 25/22; A61P 25/30; A61P 25/14; A61P 25/06; A61P 25/00; A61P 25/18

(52) U.S. Cl. 514/302; 546/115

(57) ABSTRACT

The invention relates to substituted 3-aminoisoxazolopyridines, to processes for their preparation, to medicaments containing these compounds and to the use of these compounds in the preparation of medicaments.

The invention relates to substituted 3-aminoisoxazolopyridines, to processes for their preparation, to medicaments containing these compounds and to the use of these compounds in the preparation of medicaments.

The treatment of pain, in particular of neuropathic pain, is of great importance in medicine. There is a worldwide need for effective pain therapies. The urgent need for action for a target-orientated treatment of chronic and non-chronic states of pain appropriate for the patient, by which is to be understood the successful and satisfactory treatment of pain for the patient, is also documented in the large number of scientific works which have recently been published in the field of applied analgesics and of fundamental research into nociception.

A pathophysiological feature of chronic pain is the overexcitability of neurons. Neuronal excitability is influenced decisively by the activity of K⁺ channels, since these determine decisively the resting membrane potential of the cell and therefore the excitability threshold. Heteromeric K⁺ channels of the molecular subtype KCNQ2/3 (Kv7.2/7.3) are expressed in neurons of various regions of the central (hippocampus, amygdala) and peripheral (dorsal root ganglia) nervous system and regulate the excitability thereof. Activation of KCNQ2/3 K⁺ channels leads to a hyperpolarization of the cell membrane and, accompanying this, to a decrease in the electrical excitability of these neurons. KCNQ2/3-expressing neurons of the dorsal root ganglia are involved in the transmission of nociceptive stimuli from the periphery into the spinal marrow (Passmore et al., J. Neurosci. 2003; 23(18): 7227-36).

It has accordingly been possible to detect an analgesic activity in preclinical neuropathy and inflammatory pain models for the KCNQ2/3 agonist retigabine (Blackburn-Munro and Jensen, Eur J. Pharmacol. 2003; 460(2-3); 109-16; post et al., Naunyn Schmiedebergs Arch Pharmacol 2004; 369(4): 382-390).

The KCNQ2/3 K⁺ channel thus represents a suitable starting point for the treatment of pain; in particular of pain selected from the group consisting of chronic pain, neuropathic pain, inflammatory pain and muscular pain (Nielsen et al., Eur J. Pharmacol. 2004; 487(1-3): 93-103), in particular of neuropathic and inflammatory pain.

Moreover, the KCNQ2/3 K⁺ channel is a suitable target for therapy of a large number of further diseases, such as, for example, migraine (US2002/0128277), cognitive diseases (Gribkoff, Expert Opin Ther Targets 2003; 7(6): 737-748), anxiety (Korsgaard et al., J Pharmacol Exp Ther. 2005, 14(1): 282-92), epilepsy (Wickenden et al., Expert Opin Ther Pat 2004, 14(4): 457-469; Gribkoff, Expert Opin Ther Targets 2008, 12(5): 565-81; Miceli et al., Curr Opin Pharmacol 2008, 8(1): 65-74), urinary incontinence (Streng et al., J Urol 2004; 172: 2054-2058), dependency (Hansen et al., Eur J Pharmacol 2007, 570(1-3): 77-88), mania/bipolar disorders (Dencker et al., Epilepsy Behav 2008, 12(1): 49-53), dystonia-associated dyskinesias (Richter et al., Br J Pharmacol 2006, 149(6): 747-53).

Substituted benzo[d]isoxazole derivatives which have an affinity for the KCNQ2/3 K⁺ channel are known from the prior art (WO 2006/122800).

There is a need for further compounds with comparable or better properties, not only in respect of affinity for KCNQ2/3 as such (potency, efficacy).

For example, it can be advantageous to improve the metabolic stability, the solubility in aqueous media or the permeability of the compounds. These factors can have a positive effect on the oral bioavailability or can change the PK/PD (pharmacokinetic/pharmacodynamic) profile, which can lead, for example, to a more advantageous duration of action.

A weak or non-existent interaction with transporter molecules, which are involved in the uptake and excretion of medicaments, is also to be categorized as an indication of improved bioavailability and low medicament interactions. Further, interactions with the enzymes that are involved in the degradation and excretion of medicaments should also be as low as possible, because such test results likewise indicate that low or no medicament interactions at all are to be expected.

It can also be advantageous for the compounds to exhibit a high selectivity in respect of other receptors of the KCNQ family (specificity), for example in respect of KCNQ1, KCNQ3/5 or KCNQ4. A high selectivity can have a positive effect on the side-effect profile. For example, it is known that compounds which (also) bind to KCNQ1 involve a high risk of cardiac side-effects, for which reason high selectivity in respect of KCNQ1 can be desirable. However, a high selectivity in respect of other receptors can also be advantageous. A low affinity for the hERG ion channel or for the L-type calcium ion channel (phenylalkylamine, benzothiazepine, dihydropyridine binding sites) can be advantageous because those receptors are associated with the occurrence of cardiac side-effects. Overall, an improved selectivity in respect of the binding to other endogenous proteins (i.e. e.g. receptors or enzymes) can lead to an improvement in the side-effect profile and hence to improved tolerability.

An object of the invention was, therefore, to provide novel compounds which have advantages over the compounds of the prior art. The compounds should be suitable in particular as pharmacological active ingredients in medicaments, preferably in medicaments for the treatment of disorders or diseases that are mediated at least in part by KCNQ2/3 K⁺ channels.

That object is achieved by the subject-matter of the patent claims.

Isoxazolopyridines which are suitable as modulators of the c-kit receptor are known from the prior art (WO 2008/011080 and WO 2008/011110).

It has been found, surprisingly, that substituted 3-aminoisoxazolopyridines of the general formula (1) below are suitable for the treatment of pain. It has further been found, surprisingly, that substituted 3-aminoisoxazolopyridines of the general formula (1) below also have an excellent affinity for the KCNQ2/3 K⁺ channel and are therefore suitable for the treatment of disorders or diseases that are mediated at least in part by KCNQ2/3 K⁺ channels. The substituted 3-aminoisoxazolopyridines thereby act as modulators, that is to say agonists or antagonists, of the KCNQ2/3 K⁺ channel.

The invention provides substituted 3-aminoisoxazolopyridines of the general formula (1)

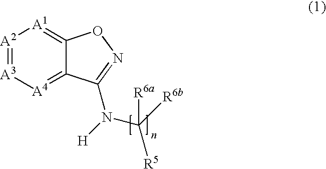

wherein
A¹ represents N or C—R¹,
A² represents N or C—R²,
A³ represents N or C—R³,
A⁴ represents N or C—R⁴,
wherein precisely one substituent A¹, A², A³ or A⁴ represents N;
R⁰ represents C₁₋₁₀-alkyl or C₂₋₁₀-heteroalkyl, in each case saturated or unsaturated, branched or unbranched, unsubstituted or mono- or poly-substituted; C₃₋₁₀-cycloalkyl or heterocyclyl, in each case saturated or unsaturated, unsubstituted or mono- or poly-substituted; aryl or heteroaryl, in each case unsubstituted or mono- or poly-substituted; C₁₋₈-alkyl- or C₂₋₈-heteroalkyl-bridged C₃₋₁₀-cycloalkyl or heterocyclyl, in each case saturated or unsaturated, unsubstituted or mono- or poly-substituted, wherein the alkyl chain or heteroalkyl chain in each case can be branched or unbranched, saturated or unsaturated, unsubstituted, mono- or poly-substituted; or C₁₋₈-alkyl- or C₂₋₈-heteroalkyl-bridged aryl or heteroaryl, in each case unsubstituted or mono- or poly-substituted, wherein the alkyl chain or heteroalkyl chain in each case can be branched or unbranched, saturated or unsaturated, unsubstituted, mono- or poly-substituted;
R¹, R², R³ and R⁴ each independently of the others denotes H; F; Cl; Br; I; NO₂; CF₃; CN; R⁰; C(═O)H; C(═O)R⁰; C(═O)OH; C(═O)OR⁰; C(═O)NH₂; C(═O)NHR⁰; C(═O)N(R⁰)₂; OH; OR⁰; O—C(═O)—R⁰; O—C(═O)—O—R⁰; O—(C═O)—NH—R⁰; O—C(═O)—N(R)₂; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SR⁰; S(═O)R⁰; S(═O)₂R⁰; S(═O)₂OH; S(═O)₂OR⁰; S(═O)₂NH₂; S(═O)₂NHR⁰; or S(═O)₂N(R⁰)₂;
or R¹ and R²; or R² and R³; or R³ and R⁴, together with the carbon atoms joining them, form an aryl or heteroaryl, in each case unsubstituted or mono- or poly-substituted;
with the proviso that R¹ may not represent NH—R⁰ when A⁴ represents N;
R⁵ represents aryl or heteroaryl, in each case unsubstituted or mono- or poly-substituted; with the proviso that when R⁵ denotes heteroaryl, unsubstituted or mono- or poly-substituted, the bonding of the heteroaryl takes place via a carbon atom of the heteroaryl;
R^{6a}, R^{6b} independently of one another represent F, Cl, Br, CN, CF₃, C₁₋₄-alkyl or O—C₁₋₄-alkyl, wherein “alkyl” within the scope of the definition of R^{6a}, R^{6b} can also be branched and/or unsaturated and/or substituted;
n represents a natural number in the range from 1 to 6, preferably from 1 to 3;
wherein “alkyl substituted”, “heteroalkyl substituted”, “heterocyclyl substituted” and “cycloalkyl substituted” denote the substitution of one or more hydrogen atoms, in each case independently of one another, by F; Cl; Br; I; CN; CF₃; ═NH; ═C(NH₂)₂; NO₂; R⁰; C(═O)H; C(═O)R⁰; CO₂H; C(═O)OR⁰; CONH₂; C(═O)NHR⁰; C(═O)N(R⁰)₂; OH; OR⁰; —O—(C₁₋₈-alkyl)-O—; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SR⁰; S(═O)R⁰; S(═O)₂R⁰; S(═O)₂H; S(═O)₂OH; S(═O)₂OR⁰; S(═O)₂NH₂; S(═O)₂NHR⁰; S(═O)₂N(R⁰)₂;
wherein “aryl substituted” and “heteroaryl substituted” denote the substitution of one or more hydrogen atoms, in each case independently of one another, by F; Cl; Br; I; NO₂; CF₃; CN; R⁰; C(═O)H; C(═O)R⁰; CO₂H; C(═O)OR⁰; CONH₂; C(═O)NHR⁰; C(═O)N(R⁰)₂; OH; OR⁰; —O—(C₁₋₈-alkyl)-O—; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SR⁰; S(═O)R⁰; S(═O)₂R⁰; S(═O)₂OH; S(═O)₂OR⁰; S(═O)₂NH₂; S(═O)₂NHR⁰; S(═O)₂N(R⁰)₂;
with the exception of the following compound:
4-methoxy-N-(2-methyl-2-phenylpropyl)isoxazolo[5,4-c]pyridin-3-amine;
in the form of the free compounds or salts of physiologically acceptable acids or bases.

Within the scope of this invention, the terms “alkyl” or “C₁₋₁₀-alkyl”, “C₁₋₈-alkyl” and “C₁₋₄-alkyl” include acyclic saturated or unsaturated aliphatic hydrocarbon radicals, which can be branched or unbranched as well as unsubstituted or mono- or poly-substituted, having from 1 to 10 or from 1 to 8 or from 1 to 4 carbon atoms, that is to say C₁₋₁₀-alkanyls, C₂₋₁₀-alkenyls and C₂₋₁₀-alkynyls or C₁₋₈-alkanyls, C₂₋₈-alkenyls and C₂₋₈-alkynyls or C₁₋₄-alkanyls, C₂₋₄-alkenyls and C₂₋₄-alkynyls. Alkenyls contain at least one C—C double bond and alkynyls contain at least one C—C triple bond. Alkyl is preferably selected from the group comprising methyl, ethyl, n-propyl, 2-propyl, n-butyl, isobutyl, sec-butyl, tert-butyl, n-pentyl, isopentyl, neopentyl, n-hexyl, n-heptyl, n-octyl, n-nonyl, n-decyl, ethenyl (vinyl), ethynyl, propenyl (—CH₂CH═CH₂, —CH═CH—CH₃, —C(═CH₂)—CH₃), propynyl (—CH—C≡CH, —C≡C—CH₃), butenyl, butynyl, pentenyl, pentynyl, hexenyl and hexynyl, heptenyl, heptynyl, octenyl, octynyl, nonenyl, nonynyl, decenyl and decynyl.

Within the scope of this invention, the terms “heteroalkyl” or “C₂₋₁₀-heteroalkyl” and “C₂₋₈-heteroalkyl” include acyclic aliphatic saturated or unsaturated hydrocarbon radicals having from 2 to 10 carbon atoms, that is to say C₂₋₁₀-heteroalkanyls, C₂₋₁₀-heteroalkenyls and C₂₋₁₀-heteroalkynyls, or having from 2 to 8 carbon atoms, that is to say C₂₋₈-heteroalkanyls, C₂₋₈-heteroalkenyls and C₂₋₈-heteroalkynyls, which in each case can be branched or unbranched as well as unsubstituted or mono- or poly-substituted and in which at least one carbon atom, optionally also two or three carbon atoms, have been replaced by a heteroatom or heteroatom group in each case selected independently of one another from the group consisting of O, N, NH and N(C₁₋₈-alkyl), preferably N(CH₃), wherein the initial carbon atom of a C₂₋₁₀-heteroalkyl or of a C₂₋₈-heteroalkyl, via which the C₂₋₁₀-heteroalkyl or C₂₋₈-heteroalkyl is bonded to the respective general structure of higher order, cannot be replaced by a heteroatom or heteroatom group and adjacent carbon atoms cannot simultaneously be replaced by a heteroatom or heteroatom group. The heteroatom groups NH and N(C₁₋₈-alkyl) of the heteroalkyl can optionally also be mono- or poly-substituted. C₂₋₁₀-Heteroalkenyls and C₂₋₈-heteroalkenyls contain at least one C—C or C—N double bond and C₂₋₁₀-heteroalkynyls and C₂₋₈-heteroalkynyls contain at least one C—C triple bond. Heteroalkyl is preferably selected from the group comprising —CH₂—O—CH₃, —CH₂—CH₂—O—CH₃, —CH₂—CH₂—O—CH₂—CH₃, —CH₂—CH₂—O—CH₂—CH₂—O—CH₃, —CH═CH—O—CH₃, —CH═CH—O—CH₂—CH₃, ═CH—O—CH₃, ═CH—O—CH₂—CH₃, ═CH—CH₂—O—CH₂—CH₃, ═CH—CH₂—O—CH₃, —CH₂—NH—CH₃, —CH₂—CH₂—NH—CH₃, —CH₂—CH₂—NH—CH₂—CH₃, —CH₂—CH₂—NH—CH₂—CH₂—NH—CH₃, —CH═CH—NH—CH₃, —CH═CH—NH—CH₂—CH₃, —CH═CH—N(CH₃)—CH₂—CH₃, ═CH—NH—CH₃, ═CH—NH—CH₂—CH₃, ═CH—CH₂—NH—CH₂—CH₃, ═CH—CH₂—NH—CH₃, —CH₂—N(CH₃)—CH₃, —CH₂—CH₂—N(CH₃)—CH₃, —CH₂—CH₂—N(CH₃)—CH₂—CH₃, —CH₂—CH₂—N(CH₃)—CH₂—CH₂—N(CH₃)—CH₃, —CH₂—CH₂—NH—CH₂—CH₂—O—CH₃, —CH₂—CH₂—O—CH₂—CH₂—NH—CH₃, —CH₂—CH₂—N(CH₃)—CH₂—CH₂—O—CH₃, —CH₂—CH₂—O—CH₂—CH₂—N(CH₃)—CH₃, —CH₂—NH—CH₂—O—CH₃, —CH₂—O—CH₂—NH—CH₃, —CH₂—N(CH₃)—CH₂—O—CH₃, —CH₂—O—CH₂—N(CH₃)—CH₃, —CH═CH—N(CH₃)—CH₃, ═CH—N(CH₃)—CH₃, ═CH—N(CH₃)—CH₂—CH₃, ═CH—CH₂—N(CH₃)—CH₂—CH₃, ═CH—CH₂—N(CH₃)—CH₃, —CH₂—CH₂═N(CH₃) and —CH₂═N(CH₃).

For the purposes of this invention, the term “cycloalkyl” or “C₃₋₁₀-cycloalkyl” denotes cyclic aliphatic hydrocarbons having 3, 4, 5, 6, 7, 8, 9 or 10 carbon atoms, wherein the hydrocarbons can be saturated or unsaturated (but not aromatic), unsubstituted or mono- or poly-substituted. The bonding of the cycloalkyl to the general structure of higher order can take place via any desired and possible ring member of the cycloalkyl radical. The cycloalkyl radicals can also be fused with further saturated, (partially) unsaturated, (hetero)cyclic, aromatic or heteroaromatic ring systems, that is to say with cycloalkyl, heterocyclyl, aryl or heteroaryl, which can themselves be unsubstituted or mono- or poly-substituted. The cycloalkyl radicals can further be bridged one or more times, as, for example, in the case of adamantyl, bicyclo[2.2.1]-heptyl or bicyclo[2.2.2]octyl. Cycloalkyl is preferably selected from the group comprising cyclopropyl, cyclobutyl, cyclopentyl, cyclohexyl, cycloheptyl, cyclooctyl, cyclononyl, cyclodecyl, adamantyl, cyclopentenyl, cyclohexenyl, cycloheptenyl and cyclooctenyl.

The term “heterocyclyl” or “heterocycloalkyl” includes aliphatic saturated or unsaturated (but not aromatic) cycloalkyls having from three to ten, that is to say 3, 4, 5, 6, 7, 8, 9 or 10, ring members, in which at least one carbon atom, optionally also two or three carbon atoms, has been replaced by a heteroatom or heteroatom group in each case selected independently of one another from the group consisting of O, S, N, NH and N(C₁₋₈-alkyl), preferably N(CH₃), wherein the ring members can be unsubstituted or mono- or poly-substituted. The bonding of the heterocyclyl to the general structure of higher order can take place via any desired and possible ring member of the heterocyclyl radical. The heterocyclyl radicals can also be fused with further saturated, (partially) unsaturated (hetero)cyclic or aromatic or heteroaromatic ring systems, that is to say with cycloalkyl, heterocyclyl, aryl or heteroaryl, which can themselves be unsubstituted or mono- or poly-substituted. Heterocyclyl radicals are preferably selected from the group comprising azetidinyl, aziridinyl, azepanyl, azocanyl, diazepanyl, dithiolanyl, dihydroquinolinyl, dihydropyrrolyl, dioxanyl, dioxolanyl, dihydroindenyl, dihydropyridinyl, dihydrofuranyl, dihydroisoquinolinyl, dihydroindolinyl, dihydroisoindolyl, imidazolidinyl, isoxazolidinyl, morpholinyl, oxiranyl, oxetanyl, pyrrolidinyl, piperazinyl, piperidinyl, pyrazolidinyl, pyranyl, tetrahydro-pyrrolyl, tetrahydropyranyl, tetrahydroquinolinyl, tetrahydroisoquinolinyl, tetrahydro-indolinyl, tetrahydrofuranyl, tetrahydropyridinyl, tetrahydrothiophenyl, tetrahydro-pyridoindolyl, tetrahydronaphthyl, tetrahydrocarbolinyl, tetrahydroisoxazolopyridinyl, thiazolidinyl and thiomorpholinyl.

Within the scope of this invention, the term “aryl” denotes aromatic hydrocarbons having up to 14 ring members, inter alia phenyls and naphthyls. Each aryl radical can be unsubstituted or mono- or poly-substituted, it being possible for the aryl substituents to be identical or different and to be in any desired and possible position of the aryl. The aryl can be bonded to the general structure of higher order via any desired and possible ring member of the aryl radical. The aryl radicals can also be fused with further saturated, (partially) unsaturated, (hetero)cyclic, aromatic or heteroaromatic ring systems, that is to say with cycloalkyl, heterocyclyl, aryl or heteroaryl, which can themselves be unsubstituted or mono- or poly-substituted. Preferred fused aryl radicals are benzodioxolanyl and benzodioxanyl. Aryl is preferably selected from the group containing phenyl, 1-naphthyl and 2-naphthyl, each of which can be unsubstituted or mono- or poly-substituted. A particularly preferred aryl is phenyl, unsubstituted or mono- or poly-substituted.

The term “heteroaryl” represents a 5- or 6-membered cyclic aromatic radical which contains at least 1 heteroatom, optionally also 2, 3, 4 or 5 heteroatoms, wherein the heteroatoms are in each case selected independently of one another from the group S, N and O and the heteroaryl radical can be unsubstituted or mono- or poly-substituted; in the case of substitution on the heteroaryl, the substituents can be identical or different and can be in any desired and possible position of the heteroaryl. Bonding to the general structure of higher order can take place via any desired and possible ring member of the heteroaryl radical. The heteroaryl can also be part of a bi- or poly-cyclic system having up to 14 ring members, wherein the ring system can be formed with further saturated, (partially) unsaturated, (hetero)cyclic or aromatic or heteroaromatic rings, that is to say with cycloalkyl, heterocyclyl, aryl or heteroaryl, which can themselves be unsubstituted or mono- or poly-substituted. It is preferred for the heteroaryl radical to be selected from the group comprising benzo-furanyl, benzimidazolyl, benzothienyl, benzothiadiazolyl, benzothiazolyl, benzo-triazolyl, benzooxazolyl, benzooxadiazolyl, quinazolinyl, quinoxalinyl, carbazolyl, quinolinyl, dibenzofuranyl, dibenzothienyl, furyl (furanyl), imidazolyl, imidazothiazolyl, indazolyl, indolizinyl, indolyl, isoquinolinyl, isoxazolyl, isothiazolyl, indolyl, naphthyridinyl, oxazolyl, oxadiazolyl, phenazinyl, phenothiazinyl, phthalazinyl, pyrazolyl, pyridyl (2-pyridyl, 3-pyridyl, 4-pyridyl), pyrrolyl, pyridazinyl, pyrimidinyl, pyrazinyl, purinyl, phenazinyl, thienyl (thiophenyl), triazolyl, tetrazolyl, thiazolyl, thiadiazolyl and triazinyl. Furyl, pyridyl and thienyl are particularly preferred.

Within the scope of the invention, the expression “C₁₋₈-alkyl-bridged aryl, heteroaryl, heterocyclyl or cycloalkyl” means that C₁₋₈-alkyl and aryl or heteroaryl or heterocyclyl or cycloalkyl have the meanings defined above and the aryl or heteroaryl or heterocyclyl or cycloalkyl radical is bonded to the structure of higher order via a C₁₋₈-alkyl group. The alkyl chain can in all cases be saturated or unsaturated, branched or unbranched, unsubstituted or mono- or poly-substituted. C₁₋₈-Alkyl is preferably selected from the group comprising —CH₂—, —CH₂—CH₂—, —CH(CH₃)—, —CH₂—CH₂—CH₂—, —CH(CH₃)—CH₂—, —CH(CH₂CH₃)—, —CH₂—(CH₂)₂—CH₂—, —CH(CH₃)—CH₂—CH₂—, —CH₂—CH(CH₃)—CH₂—, —CH(CH₃)—CH(CH₃)—, —CH(CH₂CH₃)—CH₂—, —C(CH₃)₂—CH₂—, —CH(CH₂CH₂CH₃)—, —C(CH₃)(CH₂CH₃)—, —CH₂—(CH₂)₃—CH₂—, —CH(CH₃)—CH₂—CH₂—CH₂—, —CH₂—CH(CH₃)—CH₂—CH₂—, —CH(CH₃)—CH₂—CH(CH₃)—, —CH(CH₃)—CH(CH₃)—CH₂—, —C(CH₃)₂—CH₂—CH₂—, —CH₂—C(CH₃)₂—CH₂—, —CH(CH₂CH₃)—CH₂—CH₂—, —CH₂—CH(CH₂CH₃)—CH₂—, —C(CH₃)₂—CH(CH₃)—, —CH(CH₂CH₃)—CH(CH₃)—, —C(CH₃)(CH₂CH₃)—CH₂—, —CH(CH₂CH₂CH₃)—CH₂—, —C(CH₂CH₂CH₃)—CH₂—, —CH(CH₂CH₂CH₂CH₃)—, —C(CH₃)(CH₂CH₂CH₃)—, —C(CH₂CH₃)₂—, —CH₂—(CH₂)₄—CH₂—, —CH═CH—, —CH═CH—CH₂—, —C(CH₃)═CH₂—, —CH═CH—CH₂—CH₂—, —CH₂—CH═CH—CH₂—, —CH═CH—CH═CH—, —C(CH₃)═CH—CH₂—, —CH═C(CH₃)—CH₂—, —C(CH₃)═C(CH₃)—, —C(CH₂CH₃)═CH—, —CH═CH—CH₂—CH₂—CH₂—, —CH₂—CH═CH₂—CH₂—CH₂—, —CH═CH═CH—CH₂—CH₂—, —CH═CH₂—CH—CH═CH₂—, —C≡C—, —C≡C—CH₂—, —C≡C—CH₂—CH₂—, —C≡C—CH(CH₃)—, —CH₂—C≡C—H₂—, —C≡C—C≡C—, —C≡C—C(CH₃)₂—, —C≡C—CH₂—CH₂—CH₂—, —CH₂—C≡C—CH₂—CH₂, —C≡C—C≡C—CH₂— and —C≡C—CH₂—C≡C—.

Within the scope of the invention, the expressions “C₂₋₈-heteroalkyl-bridged aryl, heteroaryl, heterocyclyl or cycloalkyl” mean that C₂₋₈-heteroalkyl and aryl or heteroaryl or heterocyclyl or cycloalkyl have the meanings defined above and the aryl or heteroaryl or heterocyclyl or cycloalkyl radical is bonded to the general structure of higher order via a C₂₋₈-heteroalkyl group. The heteroalkyl chain can in all cases be saturated or unsaturated, branched or unbranched, unsubstituted or mono- or poly-substituted. If a terminal carbon atom of the C₂₋₈-heteroalkyl group has been replaced by a heteroatom or heteroatom group, then the bonding of a heteroaryl or hetero-cyclyl to the heteroatom or heteroatom group of the C₂₋₈-heteroalkyl always takes place via a carbon atom of the heteroaryl or heterocyclyl. The terminal carbon atom is understood as being the carbon atom within the C₂₋₈-heteroalkyl that is furthest in the chain from the general structure of higher order. If the terminal carbon atom of a C₂₋₈-heteroalkyl has been replaced, for example, by an N(CH₃) group, that group is located within the C₂₋₈-heteroalkyl furthest from the general structure of higher order and is bonded to the aryl or heteroaryl or heterocyclyl or cycloalkyl radical. C₂₋₈-Heteroalkyl is preferably selected from the group comprising —CH₂—NH—, —CH₂—N(CH₃)—, —CH₂—O—, —CH₂—CH₂—NH—, —CH₂—CH₂—N(CH₃)—, —CH₂—CH₂—O—, —CH₂—CH₂—CH₂—NH—, —CH₂—CH₂—CH₂—N(CH₃)—, —CH₂—CH₂—CH₂—O—, —CH₂—O—CH₂—, —CH₂—CH₂—O—CH₂—, —CH₂—CH₂—O—CH₂—CH₂—, —CH₂—CH₂—O—CH₂—CH₂—O—CH₂—, —CH═CH—O—CH₂—, —CH═CH—O—CH₂—CH₂—, ═CH—O—CH₂—, ═CH—O—CH₂—CH₂—, ═CH—CH₂—O—CH₂—CH₂—, ═CH—CH₂—O—CH₂—, —CH₂—NH—CH₂—, —CH₂—CH₂—NH—CH₂—, —CH₂—CH₂—NH—CH₂—CH₂—, —CH₂—CH₂—NH—CH₂—CH₂—NH—CH₂, —CH═CH—NH—CH₂—, —CH═CH—NH—CH₂—CH₂—, —CH═CH—N(CH₃)—CH₂—CH₂—, ═CH—NH—CH₂—, ═CH—NH—CH₂—CH₂—, ═CH—CH₂—NH—CH₂—CH₂—, ═CH—CH₂—NH—CH₂—, —CH₂—N(CH₃)—CH₂—, —CH₂—CH₂—N(CH₃)—CH₂—, —CH₂—CH₂—N(CH₃)—CH₂—CH₂—, —CH₂—CH₂—N(CH₃)—CH₂—CH₂—N(CH₃)—CH₂—, CH₂—CH₂—NH—CH₂—CH₂—O—CH₂—, CH₂—CH₂—O—CH₂—CH₂—NH—CH₂—, CH₂—CH₂—N(CH₃)—CH₂—CH₂—O—CH₂—, CH₂—CH₂—O—CH₂—CH₂—N(CH₃)—CH₂—, CH₂—NH—CH₂—O—CH₂—, CH₂—O—CH₂—NH—CH₂—, CH₂—N(CH₃)—CH₂—O—CH₂—, CH₂—O—CH₂—N(CH₃)—CH₂—, —CH═CH—N(CH₃)—CH₂—, ═CH—N(CH₃)—CH₂—, ═CH—N(CH₃)—CH₂—CH₂—, ═CH—CH₂—N(CH₃)—CH₂—CH₂— and ═CH—CH₂—N(CH₃)—CH₂—.

In connection with “alkyl”, “heteroalkyl”, “heterocyclyl” and “cycloalkyl”, the expression “mono- or poly-substituted” is understood as meaning within the scope of this invention the substitution of one or more hydrogen atoms one or more times, for example two, three or four times, in each case independently of one another, by substituents selected from the group comprising F; Cl; Br; I; CN; CF₃; ═NH; ═C(NH₂)₂; NO₂; R⁰; C(═O)H; C(═O)R⁰; CO₂H; C(═O)OR⁰; CONH₂; C(═O)NHR⁰; C(═O)N(R⁰)₂; OH; OR⁰; —O—(C₁₋₈-alkyl)-O—; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O —S(═O)₂NHR⁰; O—S(═O)₂N(R⁰)₂; NH₂; NH—R⁰; N(R⁰)₂; NH—C(═O)—R⁰; NH—C(═O)—O—R; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SR⁰; S(═O)R⁰; S(═O)₂R⁰; S(═O)₂OH; S(═O)₂OR⁰; S(═O)₂NH₂; S(═O)₂NHR⁰; S(═O)₂N(R⁰)₂, wherein polysubstituted radicals are to be understood as being radicals that are substituted several times, for example two, three or four times, either on different atoms or on the same atom, for example three times on the same carbon atom, as in the case of CF₃ or CH₂CF₃, or at different places, as in the case of CH(OH)—CH═CH—CHCl₂. A substituent can itself optionally be mono- or poly-substituted. Polysubstitution can take place with the same or with different substituents.

Preferred “alkyl”, “heteroalkyl”, “heterocyclyl” and “cycloalkyl” substituents are selected from the group comprising F; Cl; Br; I; NO₂; CF₃; CN; ═NH; R⁰; C(═O)(R⁰ or H); C(═O)O(R⁰ or H); C(═O)N(R⁰ or H)₂; OH; OR⁰; O—C(═O)—R⁰; O—(C₁₋₈-alkyl)-OH; —O—(C₁₋₈-alkyl)-O—; O—(C₁₋₈-alkyl)-O—C₁₋₈-alkyl; OCF₃; N(R⁰ or H)₂; N(R⁰ or H)—C(═O)—R⁰; N(R⁰ or H)—C(═O)—N(R⁰ or H)₂; SH; SCF₃; SR⁰; S(═O)₂R⁰; S(═O)₂O(R⁰ or H) and S(═O)₂—N(R⁰ or H)₂.

The expression “(R⁰ or H)” within a radical means that R⁰ and H can occur in any possible combination within that radical. For example, the radical “N(R⁰ or H)₂” can represent “NH₂”, “NHR⁰” and)“N(R⁰)₂”. When R⁰ occurs several times within a radical, as in the case of)“N(R⁰)₂”, then R⁰ can have identical or different meanings; in the present example of)“N(R⁰)₂”, R⁰ can twice represent aryl, for example, yielding the functional group “N(aryl)₂”, or R⁰ can once represent aryl and once represent C₁₋₁₀-alkyl, yielding the functional group “N(aryl)(C₁₋₁₀-alkyl)”.

Particularly preferred “alkyl”, “heteroalkyl”, “heterocyclyl” and “cycloalkyl” substituents are selected from the group consisting of F; Cl; Br; I; NO₂; CF₃; CN; C₁₋₈-alkyl; aryl; heteroaryl; C₃₋₁₀-cycloalkyl; heterocyclyl; C₁₋₈-alkyl-bridged aryl, heteroaryl, C₃₋₁₀-cycloalkyl or heterocyclyl; CHO; C(═O)C₁₋₈-alkyl; C(═O)aryl; C(═O)heteroaryl; CO₂H; C(═O)O—C₁₋₈-alkyl; C(═O)O-aryl; C(═O)O-heteroaryl; CONH₂; C(═O)NH—C₁₋₈-alkyl; C(═O)N(C₁₋₈-alkyl)₂; C(═O)NH-aryl; C(═O)N(aryl)₂; C(═O)NH-heteroaryl; C(═O)N(heteroaryl)₂; C(═O)N(C₁₋₈-alkyl)(aryl); C(═O)N(C₁₋₈-alkyl)(heteroaryl); C(═O)N(heteroaryl)(aryl); OH; O—C₁₋₈-alkyl; OCF₃; O—(C₁₋₈-alkyl)-OH; O—(C₁₋₈-alkyl)-O—C₁₋₈-alkyl; O-benzyl; O-aryl; O-heteroaryl; O—C(═O)C₁₋₈-alkyl; O—C(═O)aryl; O—C(═O)heteroaryl; NH₂; NH—C₁₋₈-alkyl; N(C₁₋₈-alkyl)₂; NH—C(═O)C₁₋₈-alkyl; NH—C(═O)-aryl; NH—C(═O)-heteroaryl; SH; S—C₁₋₈-alkyl; SCF₃; S-benzyl; S-aryl; S-heteroaryl; S(═O)₂C₁₋₈-alkyl; S(═O)₂aryl; S(═O)₂heteroaryl; S(═O)₂OH; S(═O)₂O—C₁₋₈-alkyl; S(═O)₂O-aryl; S(═O)₂O-heteroaryl; S(═O)₂—NH—C₁₋₈-alkyl; S(═O)₂—NH-aryl; and S(═O)₂—NH—C₁₋₈-heteroaryl.

In connection with “aryl” and “heteroaryl”, “mono- or poly-substituted” is understood within the scope of this invention as meaning the substitution of one or more hydrogen atoms of the ring system one or more times, for example two, three or four times, in each case independently of one another, by substituents selected from the group comprising F; Cl; Br; I; NO₂; CF₃; CN; R⁰; C(═O)H; C(═O)R⁰; CO₂H; C(═O)OR⁰; CONH₂; C(═O)NHR⁰; C(═O)N(R⁰)₂; OH; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SR⁰; S(═O)R⁰; S(═O)₂R⁰; S(═O)₂OH; S(═O)₂OR⁰; S(═O)₂NH₂; S(═O)₂NHR⁰; S(═O)₂N(R⁰)₂, on one atom or optionally on different atoms, wherein a substituent can itself optionally be mono- or poly-substituted. Polysubstitution is carried out with the same or with different substituents.

Preferred “aryl” and “heteroaryl” substituents are F; Cl; Br; I; NO₂; CF₃; CN; R⁰; C(═O)(R⁰ or H); C(═O)O(R⁰ or H); C(═O)N(R⁰ or H)₂; OH; OR⁰; O—C(═O)—R⁰; —O—(Cl_{—}8-alkyl)-O—; O—(C₁₋₈-alkyl)-O—C₁₋₈-alkyl; OCF₃; N(R⁰ or H)₂; N(R⁰ or H)—C(═O)—R⁰; N(R⁰ or H)—C(═O)—N(R⁰ or H)₂; SH; SCF₃; SR⁰; S(═O)₂R⁰; S(═O)₂O(R⁰ or H); S(═O)₂—N(R⁰ or H)₂.

Particularly preferred “aryl” and “heteroaryl” substituents are selected from the group consisting of F; Cl; Br; I; NO₂; CF₃; CN; C₁₋₈-alkyl; aryl; heteroaryl; C₃₋₁₀-cycloalkyl; heterocyclyl; C₁₋₈-alkyl-bridged aryl, heteroaryl, C₃₋₁₀-cycloalkyl or heterocyclyl; CHO; C(═O)C₁₋₈-alkyl; C(═O)aryl; C(═O)heteroaryl; CO₂H; C(═O)O—C₁₋₈-alkyl; C(═O)O-aryl; C(═O)O-heteroaryl; CONH₂; C(═O)NH—C₁₋₈-alkyl; C(═O)N(C₁₋₈-alkyl)₂; C(═O)NH-aryl; C(═O)N(aryl)₂; C(═O)NH-heteroaryl; C(═O)N(heteroaryl)₂; C(═O)N(C₁₋₈-alkyl)(aryl); C(═O)N(C₁₋₈-alkyl)(heteroaryl); C(═O)N(heteroaryl)(aryl); OH; O—C₁₋₈-alkyl; OCF₃; —O—(C₁₋₈-alkyl)-O—; O—(C₁₋₈-alkyl)-OH; O—(C₁₋₈-alkyl)-O—C₁₋₈-alkyl; O-benzyl; O-aryl; O-heteroaryl; O—C(═O)C₁₋₈-alkyl; O—C(═O)aryl; O—C(═O)heteroaryl; NH₂ NH—C₁₋₈-alkyl; N(C₁₋₈-alkyl)₂; NH—C(═O)C₁₋₈-alkyl; NH—C(═O)-aryl; NH—C(═O)-heteroaryl; SH; S—C₁₋₈-alkyl; SCF₃; S-benzyl; S-aryl; S-heteroaryl; S(═O)₂C₁₋₈-alkyl; S(═O)₂aryl; S(═O)₂heteroaryl; S(═O)₂OH; S(═O)₂O—C₁₋₈-alkyl; S(═O)₂O-aryl; S(═O)₂O-heteroaryl; S(═O)₂—NH—C₁₋₈-alkyl; S(═O)₂—NH-aryl; S(═O)₂—NH—C₁₋₈-heteroaryl.

The compounds according to the invention are defined by substituents, for example by R¹, R² and R³ (1st generation substituents), which are themselves optionally substituted (2nd generation substituents). Depending on the definition, these substituents of the substituents can in turn themselves be substituted (3rd generation substituents). If, for example, R³═R⁰, wherein R⁰=aryl (1st generation substituent), aryl can itself be substituted, for example by NHR⁰, wherein R⁰═C₁₋₁₀-alkyl (2nd generation substituent). This yields the functional group aryl-NHC₁₋₁₀-alkyl. C₁₋₁₀-Alkyl can then in turn itself be substituted, for example by Cl (3rd generation substituent). Overall, this then yields the functional group aryl-NHC₁₋₁₀-alkyl-Cl.

In a preferred embodiment, however, the 3rd generation substituents cannot themselves be substituted, that is to say there are no 4th generation substituents.

In another preferred embodiment, the 2nd generation substituents cannot themselves be substituted, that is to say there are not even any 3rd generation substituents. In other words, the functional groups for R⁰ to R⁵ in each case can optionally be substituted in this embodiment, for example in the case of the general formula (1), but the substituents cannot themselves be substituted.

If a radical occurs more than once within a molecule, such as, for example, the radical R⁰, that radical can have different meanings for different substituents: if, for example, both R¹═R⁰ and R²═R⁰, R⁰ can denote aryl for R¹ and C₁₋₁₀-alkyl for R².

In some cases, the compounds according to the invention are defined by substituents which are or carry an aryl or heteroaryl radical, in each case unsubstituted or mono- or poly-substituted, or which, together with the carbon atom(s) or heteroatom(s) joining them as ring member(s), form a ring, for example an aryl or heteroaryl, in each case unsubstituted or mono- or poly-substituted. Both these aryl or heteroaryl radicals and the aromatic ring systems so formed can optionally be fused with C₃-C₁₀-cycloalkyl or heterocyclyl, in each case saturated or unsaturated, that is to say with a C₃₋₁₀-cycloalkyl such as cyclopentyl or with a heterocyclyl such as morpholinyl, it being possible for the C₃₋₁₀-cycloalkyl or heterocyclyl radicals so fused to be unsubstituted or mono- or poly-substituted.

In some cases, the compounds according to the invention are defined by substituents which are or carry a C₃₋₁₀-cycloalkyl or heterocyclyl radical, in each case unsubstituted or mono- or poly-substituted, or which, together with the carbon atom(s) or heteroatom(s) joining them as ring member(s), form a ring, for example a C₃₋₁₀-cycloalkyl or heterocyclyl, in each case unsubstituted or mono- or poly-substituted. Both these C₃₋₁₀-cycloalkyl or heterocyclyl radicals and the aliphatic ring systems so formed can optionally be fused with aryl or heteroaryl, that is to say with an aryl such as phenyl or with a heteroaryl such as pyridyl, it being possible for the aryl or heteroaryl radicals so fused to be unsubstituted or mono- or poly-substituted.

Within the scope of the present invention, the symbol

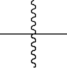

used in formulae denotes a linking of a corresponding radical to the general structure of higher order.

The expression “salt formed with a physiologically acceptable acid” is understood within the scope of this invention as meaning salts of the active ingredient in question with inorganic or organic acids that are physiologically acceptable—in particular when used in humans and/or mammals. The hydrochloride is particularly preferred.

Examples of physiologically acceptable acids are: hydrochloric acid, hydrobromic acid, sulfuric acid, methanesulfonic acid, formic acid, acetic acid, oxalic acid, succinic acid, tartaric acid, mandelic acid, fumaric acid, maleic acid, lactic acid, citric acid, glutamic acid, saccharinic acid, monomethylsebacic acid, 5-oxo-proline, hexane-1-sulfonic acid, nicotinic acid, 2-, 3- or 4-aminobenzoic acid, 2,4,6-trimethyl-benzoic acid, α-liponic acid, acetylglycine, hippuric acid, phosphoric acid and/or aspartic acid. Citric acid and hydrochloric acid are particularly preferred.

Physiologically acceptable salts with cations or bases are salts of the compound in question—in the form of the anion with at least one, preferably inorganic cation—that are physiologically acceptable—in particular when used in humans and/or mammals. Particular preference is given to the salts of the alkali and alkaline earth metals but also to ammonium salts, but in particular to (mono-) or (di-)sodium, (mono-) or (di-)potassium, magnesium or calcium salts.

Preferred embodiments of the compounds of the general formula (1) according to the invention have the general formula (1a), (1b), (1c) or (1d):

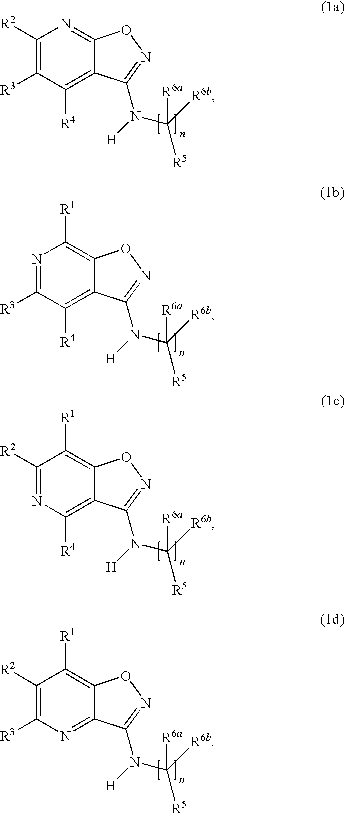

Compounds of the general formulae (1a) and (1b) are particularly preferred.

In a preferred embodiment, the substituents R¹, R², R³ and R⁴, each independently of the others, are selected from the group consisting of H; F; Cl; Br; I; NO₂; CF₃; CN; R⁰; C(═O)(R⁰ or H); C(═O)O(R⁰ or H); C(═O)N(R⁰ or H)₂; OH; OR⁰; O—(C₁₋₈-alkyl)-OH; O—(C₁₋₈-alkyl)-O—C₁₋₈-alkyl; OCF₃; N(R⁰ or H)₂; N(R⁰ or H)—C(═O)—R⁰; N(R⁰ or H)—C(═O)—N(R⁰ or H)₂; SH; SCF₃; SR⁰; S(═O)₂R⁰; S(═O)₂O(R⁰ or H); and S(═O)₂—N(R⁰ or H)₂;

or R¹ and R²; or R² and R³; or R³ and R⁴, together with the carbon atoms joining them, form an aryl or heteroaryl, in each case unsubstituted or mono- or poly-substituted;
with the proviso that R¹ may not represent NH—R⁰, C(═O)N(R⁰ or H)₂, N(R⁰ or H)—C(═O)—R⁰, N(R⁰ or H)—C(═O)—N(R⁰ or H)₂ or S(═O)₂—N(R⁰ or H)₂ when A⁴ represents N.

In a further preferred embodiment, R⁴ denotes H; F; Cl; Br; I; NO₂; CF₃; CN; R⁰; C(═O)H; C(═O)R⁰; C(═O)OH; C(═O)OR⁰; C(═O)NH₂; C(═O)NHR⁰; C(═O)N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SR⁰; S(═O)R⁰; S(═O)₂R⁰; S(═O)₂OH; S(═O)₂OR⁰; S(═O)₂NH₂; S(═O)₂NHR⁰; or S(═O)₂N(R⁰)₂.

In a further preferred embodiment, R^{6a}, R^{6b} independently of one another denote methyl or ethyl which can be substituted by a group selected from F; Cl; Br; I; CF₃; —O—CH₃, CN.

In a further preferred embodiment, n is 1, 2 or 3, particularly preferably 1.

In another preferred embodiment, R¹ represents H; F; Cl; Br; I; NO₂; CF₃; CN; R⁰; C(═O)H; C(═O)R⁰; C(═O)OH; C(═O)OR⁰; C(═O)NH₂; C(═O)NHR⁰; C(═O)N(R⁰)₂; OH; OR⁰; O—C(═O)—R⁰; O—C(═O)—O—R⁰; O—(C═O)—NH—R⁰; O—C(═O)—N(R⁰)₂; O—S(═O)₂OH; O—S(═O)₂OR⁰; O—S(═O)₂NH₂; O—S(═O)₂NHR⁰; O—S(═O)₂N(R⁰)₂; SH; SR⁰; S(═O)R⁰; S(═O)₂R⁰; S(═O)₂OH; S(═O)₂OR⁰; S(═O)₂NH₂; S(═O)₂NHR⁰; or S(═O)₂N(R⁰)₂.

In a further preferred embodiment, R¹ and R² each independently of the other represents H; F; Cl; Br; I; NO₂; CN; OH; O—C₁₋₈-alkyl; O—(C₁₋₈-alkyl)-OH; O—(C₁₋₈-alkyl)-O—C₁₋₈-alkyl; OCF₃; C₁₋₈-alkyl; C(═O)—OH; CF₃; NH₂; NH(C₁₋₈-alkyl); N(C₁₋₈-alkyl)₂; SH; S—C₁₋₈-alkyl; SCF₃; S(═O)₂OH; benzyl, aryl or heteroaryl, in each case unsubstituted or mono- or poly-substituted by one or more substituents selected independently of one another from the group consisting of F, Cl, Br, I, CN, OH, O—C₁₋₈-alkyl, OCF₃, C₁₋₈-alkyl, CF₃, SCF₃;

and R³ and R⁴ each independently of the other represents H; F; Cl; Br; I; NO₂; CN; OH; O—C₁₋₈-alkyl; O—(C₁₋₈-alkyl)-OH; O—(C₁₋₈-alkyl)-O—C₁₋₈-alkyl; OCF₃; C₁₋₈-alkyl; C(═O)—OH; CF₃; NH₂; NH(C₁₋₈-alkyl); N(C₁₋₈-alkyl)₂; SH; S—C₁₋₈-alkyl; SCF₃; S(═O)₂OH; C(═O)NH₂; C(═O)NH(C₁₋₈-alkyl); C(═O)N(C₁₋₈-alkyl)₂; C(═O)NH(aryl); C(═O)N(aryl)₂; C(═O)NH(heteroaryl); C(═O)N(heteroaryl)₂; C(═O)N(C₁₋₈-alkyl)(aryl); C(═O)N(C₁₋₈-alkyl)(heteroaryl); C(═O)N(heteroaryl)(aryl); NH—C(═O)—C₁₋₈-alkyl; NH—C(═O)-aryl; NH—C(═O)-heteroaryl; N(C₁₋₈-alkyl)-C(═O)—C₁₋₈-alkyl; N(C₁₋₈-alkyl)-C(═O)-aryl; N(C₁₋₈-alkyl)-C(═O)-heteroaryl; N(aryl)-C(═O)—C₁₋₈-alkyl; N(aryl)-C(═O)-aryl; N(heteroaryl)-C(═O)-heteroaryl; NH—C₁₋₈-alkyl-heterocyclyl; NH—C₁₋₈-alkyl-cycloalkyl; NH—C₁₋₈-alkyl-aryl; NH—C₁₋₈-alkyl-heteroaryl; N(C₁₋₈-alkyl)-C₁₋₈-alkyl-heterocyclyl; N(C₁₋₈-alkyl)-C₁₋₈-alkyl-cycloalkyl; N(C₁₋₈-alkyl)-C₁₋₈-alkyl-aryl; N(C₁₋₈-alkyl)-C₁₋₈-alkyl-heteroaryl; NH—C(═O)C₁₋₈-alkyl-heterocyclyl; NH—C(═O)C₁₋₈-alkyl-cycloalkyl; NH—C(═O)C₁₋₈-alkyl-aryl; NH—C(═O)C₁₋₈-alkyl-heteroaryl; N(C₁₋₈-alkyl)-C(═O)C₁₋₈-alkyl-heterocyclyl; N(C₁₋₈-alkyl)-C(═O)C₁₋₈-alkyl-cycloalkyl; N(C₁₋₈-alkyl)-C(═O)C₁₋₈-alkyl-aryl; N(C₁₋₈-alkyl)-C(═O)—C₁₋₈-alkyl-heteroaryl; benzyl; wherein aryl or heteroaryl can in each case be unsubstituted or mono- or poly-substituted by one or more substituents selected independently of one another from the group consisting of F, Cl, Br, I, CN, OH, O—C₁₋₈-alkyl, OCF₃, C₁₋₈-alkyl, CF₃, SCF₃; and wherein cycloalkyl or heterocyclyl can in each case be unsubstituted or mono- or poly-substituted by one or more substituents selected independently of one another from the group consisting of F, Cl, Br, I, CN, OH, O—C₁₋₈-alkyl, OCF₃, C₁₋₈-alkyl, CF₃, SCF₃.

Preferably, R¹, R², R³ and R⁴ each independently of the others represents H; F; Cl; Br; I; NO₂; CN; OH; O—C₁₋₈-alkyl; O—(C₁₋₈-alkyl)-OH; O—(C₁₋₈-alkyl)-O—C₁₋₈-alkyl; OCF₃; C₁₋₈-alkyl; C(═O)—OH; CF₃; NH₂; NH(C₁₋₈-alkyl); N(C₁₋₈-alkyl)₂; SH; S—C₁₋₈-alkyl; SCF₃; S(═O)₂OH; benzyl, phenyl, in each case unsubstituted;

or R¹ and R²; or R² and R³; or R³ and R⁴, together with the carbon atoms joining them, form a phenyl, in each case unsubstituted or mono- or poly-substituted by one or more substituents selected independently of one another from the group consisting of F, Cl, Br, I, CN, OH, O—C₁₋₈-alkyl, OCF₃, C₁₋₈-alkyl, CF₃, SCF₃.

Particularly preferably, R¹, R², R³ and R⁴ each independently of the others represents H; F; Cl; Br; I; CN; O—C₁₋₈-alkyl; OCF₃; C₁₋₈-alkyl; CF₃; NH₂; S—C₁₋₈-alkyl; SCF₃;

or R¹ and R²; or R² and R³; or R³ and R⁴, together with the carbon atoms joining them, form an unsubstituted phenyl.

Most particularly preferably, R¹ and R² each independently of the other represents H; F; Cl; Br; I; O—C₁₋₈-alkyl; C₁₋₈-alkyl; OCF₃; CF₃;

and R³ and R⁴ each independently of the other represents H; F; Cl; Br; I; S—C₁₋₈-alkyl; SCF₃;
or R² and R³, together with the carbon atoms joining them, form an unsubstituted phenyl.

In particular, R¹ and R² each independently of the other represents H; C₁₋₈-alkyl; CF₃;

R³ represents H;
and R⁴ represents H; F; Cl; Br; I; S—C₁₋₈-alkyl;
or R² and R³, together with the carbon atoms joining them, form an unsubstituted phenyl.

In a further preferred embodiment, R⁵ represents aryl or heteroaryl, in each case unsubstituted or mono- or poly-substituted by one or more substituents selected independently of one another from the group consisting of F, Cl, Br, I, NO₂, CN, OH, O—C₁₋₈-alkyl, OCF₃, C₁₋₈-alkyl, C(═O)—OH, CF₃, NH₂, NH(C₁₋₈-alkyl), N(C₁₋₈-alkyl)₂, SH, S—C₁₋₈-alkyl, SCF₃, S(═O)₂OH, benzyl, unsubstituted, and phenyl,

wherein aryl or heteroaryl in each case can optionally be fused with C₃₋₁₀-cycloalkyl or heterocyclyl, in each case saturated or unsaturated, unsubstituted or mono- or poly-substituted by one or more substituents selected independently of one another from the group consisting of F, Cl, Br, I, NO₂, CN, OH, O—C₁₋₈-alkyl, OCF₃, C₁₋₈-alkyl, C(═O)—OH, CF₃, NH₂, NH(C₁₋₈-alkyl), N(C₁₋₈-alkyl)₂, SH, S—C₁₋₈-alkyl, SCF₃, S(═O)₂OH, benzyl, unsubstituted, and phenyl, unsubstituted.

Preferably, R⁵ represents phenyl, pyridyl or thienyl, in each case unsubstituted or mono- or poly-substituted by one or more substituents selected independently of one another from the group consisting of F, Cl, Br, I, NO₂, CN, OH, O—C₁₋₈-alkyl, OCF₃, C₁₋₈-alkyl, C(═O)—OH, CF₃, NH₂, NH(C₁₋₈-alkyl), N(C₁₋₈-alkyl)₂, SH, S—C₁₋₈-alkyl, SCF₃, S(═O)₂OH;

wherein phenyl and pyridyl in each case can optionally be fused with C₃₋₁₀-cycloalkyl, saturated or unsaturated, unsubstituted or mono- or poly-substituted by one or more substituents selected independently of one another from the group consisting of F, Cl, Br, I, NO₂, CN, OH, O—C₁₋₈-alkyl, OCF₃, C₁₋₈-alkyl, C(═O)—OH, CF₃, NH₂, NH(C₁₋₈-alkyl), N(C₁₋₈-alkyl)₂, SH, S—C₁₋₈-alkyl, SCF₃, S(═O)₂OH and benzyl, unsubstituted; or phenyl and pyridyl in each case can optionally be fused with heterocyclyl, saturated or unsaturated, in which one or two carbon atoms have been replaced by a heteroatom in each case selected independently of one another from the group consisting of O, N, NH and N(C₁₋₈-alkyl), in each case unsubstituted or mono- or poly-substituted by one or more substituents selected independently of one another from the group consisting of F, Cl, Br, I, NO₂, CN, OH, O—C₁₋₈-alkyl, OCF₃, C₁₋₈-alkyl, C(═O)—OH, CF₃, NH₂, NH(C₁₋₈-alkyl), N(C₁₋₈-alkyl)₂, SH, S—C₁₋₈-alkyl, SCF₃, S(═O)₂OH and benzyl, unsubstituted.

Particular preference is given to compounds having the general formula (2)

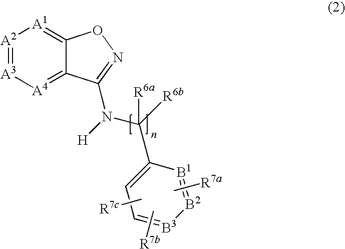

wherein
B¹ represents CH or N;
B² represents CH or N;
B³ represents CH or N;
wherein not more than one of the substituents B¹, B² and B³ can represent N;
   R^{7a}, R^{7b} and R^{7c} each independently of the others represents H; F; Cl; Br; I; CN; OH; O—C₁₋₈-alkyl; OCF₃; C₁₋₈-alkyl; CF₃; SCF₃;
   or the substituents R^{7a} and R^{7b}, together with the carbon atoms joining them, form one of the following partial structures I-a, I-b, I-c or I-d:

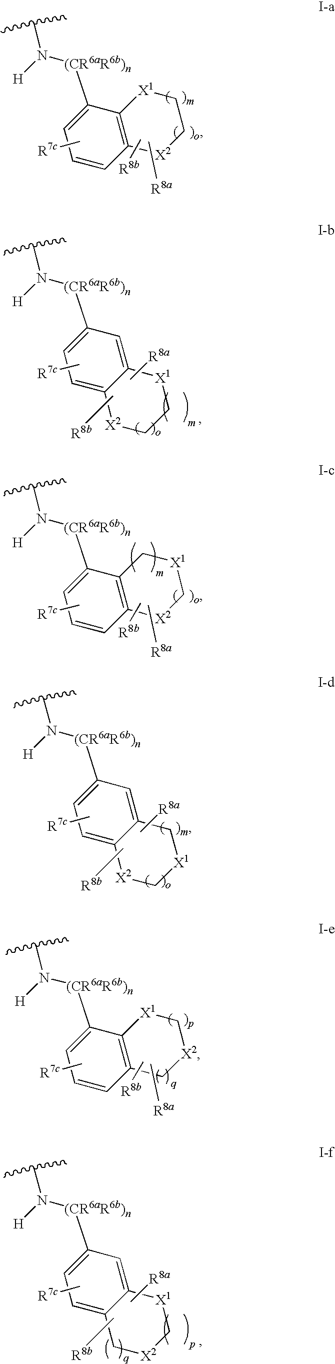

wherein
m in each case represents 0, 1 or 2;
o in each case represents 1, 2 or 3;
p in each case represents 1, 2 or 3;
q in each case represents 0, 1 or 2;
X¹ and X² each independently of the other represents CH₂, O, NH or N(C₁₋₈-alkyl);
R^{8a} and R^{8b} each independently of the other represents F, Cl, Br, I, NO₂, CN, OH, O—C₁₋₈-alkyl, OCF₃, C₁₋₈-alkyl, CF₃, NH₂, NH(C₁₋₈-alkyl) and N(C₁₋₈-alkyl)₂;
and the remaining substituents R^{8a}, R^{6b} and R^{7c} have one of the meanings given above.

Particular preference is given to compounds of the general formula (2)

wherein
n represents 1 or 2; preferably 1;
R^{6a} and R^{6b} each independently of the other represents H, F, Cl, Br, I, C₁₋₈-alkyl, saturated, unsaturated, branched or unbranched, unsubstituted;
R^{7a}, R^{7b} and R^{7c} each independently of the others represents H; F; Cl; Br; I; O—C₁₋₈-alkyl; OCF₃; C₁₋₈-alkyl; CF₃; SCF₃;
or the substituents R^{7a} and R^{7b}, together with the carbon atoms joining them, form one of the following partial structures:

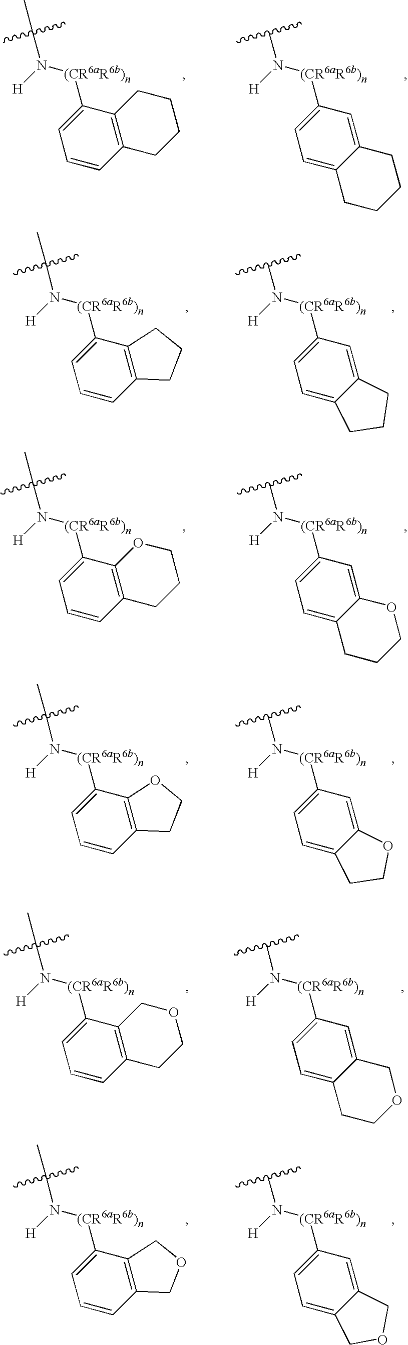

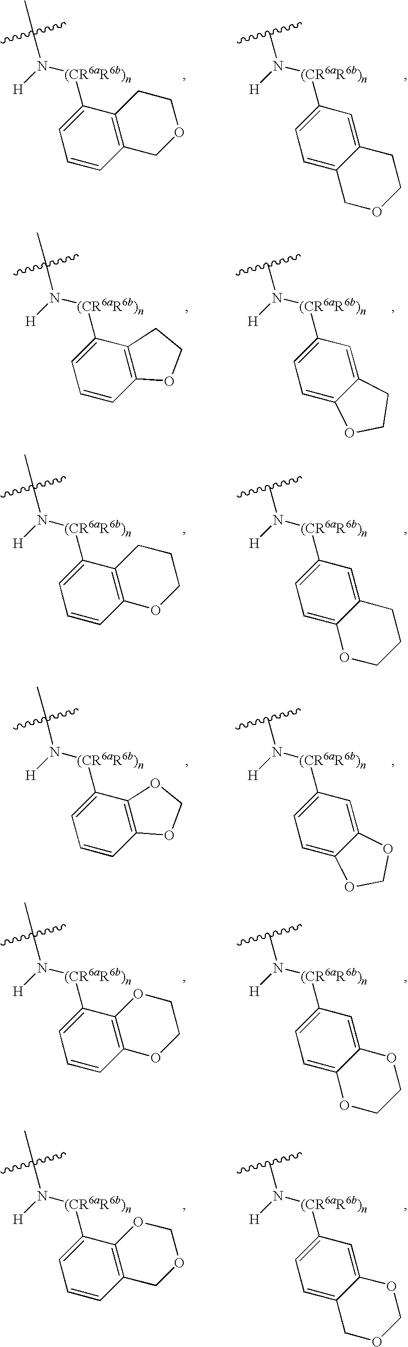

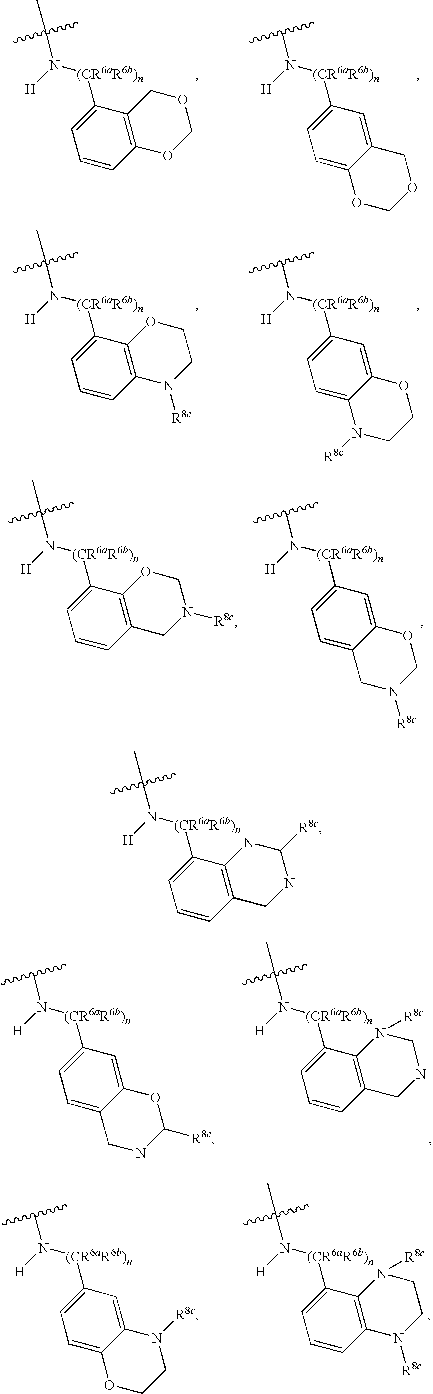

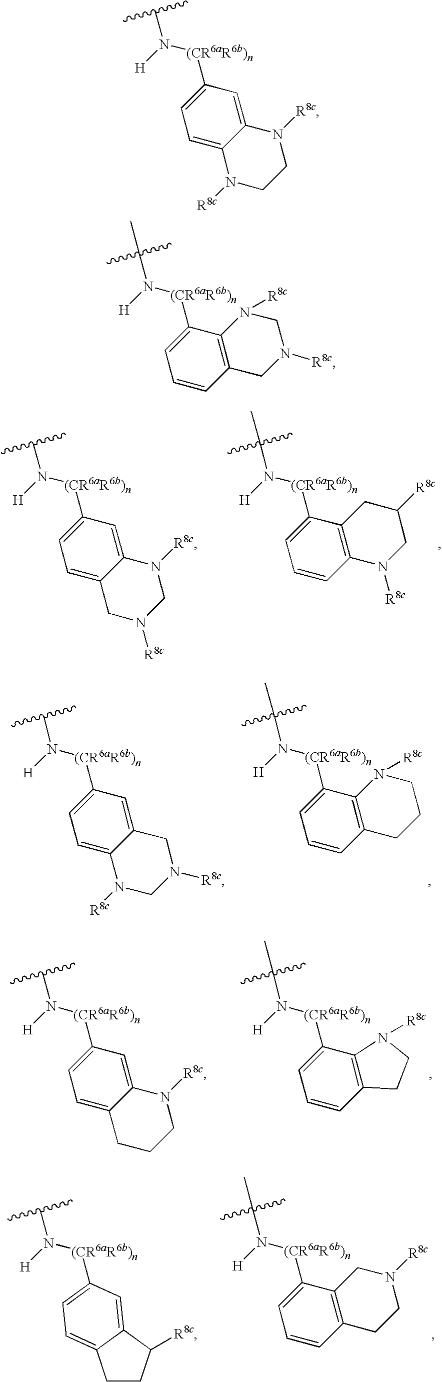

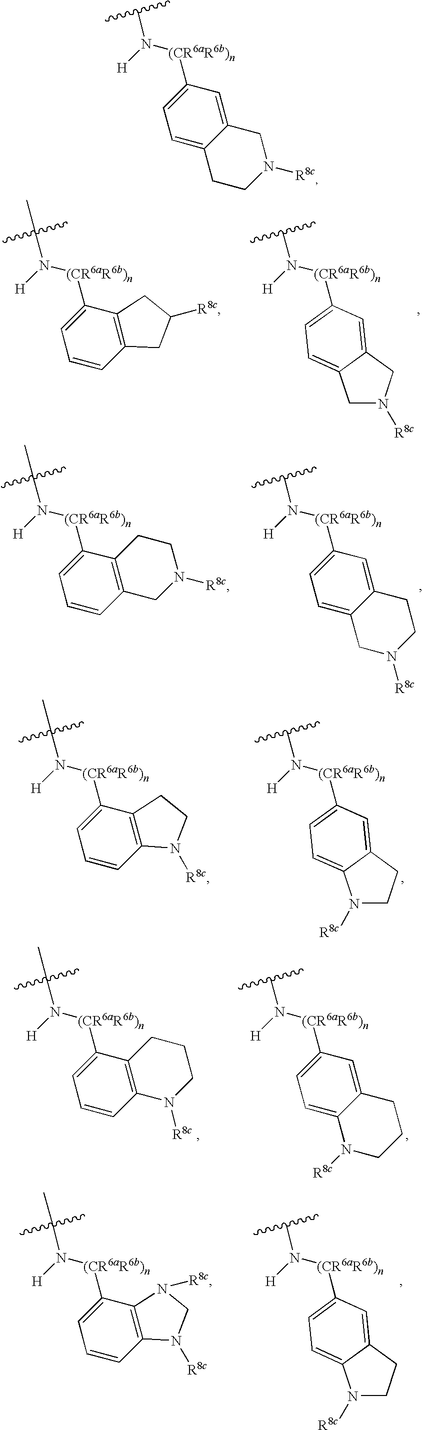

wherein Rbc in each case represents H or C₁₋₈-alkyl.

Most particular preference is given to compounds of the general formula (1) according to the invention wherein

R¹, R², R³ and R⁴ each independently of the others denotes H; F; Cl; Br; I; CN; O—C₁₋₈-alkyl; OCF₃; C₁₋₈-alkyl; CF₃; NH₂; S—C₁₋₈-alkyl; SCF₃;
or R¹ and R²; or R² and R³; or R³ and R⁴, together with the carbon atoms joining them, form an unsubstituted phenyl;

In a further preferred embodiment, R⁵ represents aryl or heteroaryl, in each case unsubstituted or mono- or poly-substituted by one or more substituents selected independently of one another from the group consisting of F, Cl, Br, I, NO₂, CN, OH, O—C₁₋₈-alkyl, OCF₃, C₁₋₈-alkyl, C(═O)—OH, CF₃, NH₂, NH(C₁₋₈-alkyl), N(C₁₋₈-alkyl)₂, SH, S—C₁₋₈-alkyl, SCF₃, S(═O)₂OH, benzyl, unsubstituted, and phenyl.

Particular preference is given to compounds from the group
1 N-(1-(4-(trifluoromethyl)phenyl)ethyl)-isoxazolo[5,4-b]pyridin-3-amine;
2 N-(4-(trifluoromethylthio)benzypisoxazolo-[5,4-b]pyridin-3-amine;
3 N-(3,5-dimethyl-benzyl)isoxazolo[5,4-b]pyridin-3-amine;
4 N-(1-(3,5-dimethyl-phenyl)ethypisoxazolo[5,4-b]pyridin-3-amine;
5 N-(1-(4-(trifluoromethylthio)phenyl)ethyl)-isoxazolo[5,4-b]pyridin-3-amine;
6 N-(4-(trifluoromethylthio)benzypisoxazolo[5,4-c]pyridin-3-amine;
7 N-(3,5-dimethyl-benzyl)isoxazolo[5,4-c]pyridin-3-amine;
8 N-(3,5-dimethyl-benzyl)isoxazolo[4,5-b]pyridin-3-amine;
9 N-(4-(trifluoromethylthio)benzypisoxazolo[4,5-b]pyridin-3-amine;
10 N-(1-(3,5-dimethyl-phenyl)ethypisoxazolo[4,5-b]pyridin-3-amine;
11 N-(3,5-dimethyl-benzyl)isoxazolo[4,5-c]pyridin-3-amine;
12 N-(4-(trifluoromethylthio)benzypisoxazolo[4,5-c]pyridin-3-amine;
13 N-(1-(4-(trifluoromethylthio)phenyl)ethypisoxazolo[4,5-b]pyridin-3-amine;
14 N-(1-(4-(trifluoromethyl)phenyl)ethypisoxazolo[5,4-c]pyridin-3-amine;
15 N-(1-(3,5-dimethyl-phenyl)ethypisoxazolo[5,4-c]pyridin-3-amine;
16 N-(1-(3,5-dimethyl-phenyl)ethypisoxazolo[4,5-c]pyridin-3-amine;
17 N-(1-(4-(trifluoromethylthio)phenyl)ethyl)-isoxazolo[4,5-c]pyridin-3-amine;
18 N-(1-(3,5-dimethylphenyl)ethyl)-6-(trifluoromethypisoxazolo[5,4-b]pyridin-3-amine;
19 N-(4-(trifluoromethylthio)benzyl)-6-trifluoromethyl-isoxazolo[5,4-b]pyridin-3-amine;
20 6-methyl-N-(4-(trifluoromethylthio)benzyl)-isoxazolo[5,4-b]pyridin-3-amine;
21 N-(1-(3,5-dimethyl-phenyl)ethyl)-7-trifluoromethyl-isoxazolo[5,4-c]pyridin-3-amine;
22 N-(1-(3,5-dimethylphenyl)ethyl)-6-methyl-isoxazolo[5,4-b]pyridin-3-amine;
23 N-(4-(trifluoromethylthio)benzyl)-7-trifluoromethyl-isoxazolo[5,4-c]pyridin-3-amine;
24 N-(4-(trifluoromethylthio)benzypisoxazolo[5,4-b]quinolin-3-amine;
25 4-chloro-N-(4-(trifluoromethylthio)benzyl)-isoxazolo[4,5-c]pyridin-3-amine;
26 4-pentylthio-N-(4-(trifluoromethylthio)-benzyl)isoxazolo[4,5-c]pyridin-3-amine;
27 N-(6-chloropyridin-3-yl)isoxazolo[5,4-b]pyridin-3-amine;
28 N-(6-chloropyridin-3-yl)-6-(trifluoromethyl)isoxazolo[5,4-b]pyridin-3-amine;
29 N-(6-chloropyridin-3-yl)isoxazolo[5,4-c]pyridin-3-amine;
30 N-(6-chloropyridin-3-yl)-7-(trifluoromethypisoxazolo[5,4-c]pyridin-3-amine;
31 N-phenyl-6-(trifluoromethyl)isoxazolo[5,4-b]pyridin-3-amine;
32 N-phenyl-7-(trifluoromethyl)isoxazolo[5,4-c]pyridin-3-amine;
   or physiologically acceptable salts thereof.

The substituted 3-aminoisoxazolopyridines according to the invention and in each case the corresponding acids, bases, salts and solvates are suitable as pharmaceutical active ingredients in medicaments.

The invention therefore further provides a medicament comprising at least one substituted 3-aminoisoxazolopyridine of the general formula (1) according to the invention wherein the radicals R¹ to R⁵ have the meaning given above and, optionally, one or more pharmaceutically acceptable auxiliary substances.

In addition to at least one compound according to the invention, the medicaments according to the invention optionally comprise suitable additives and/or auxiliary substances, that is to say also carriers, fillers, solvents, diluents, colourings and/or binders, and can be administered as liquid medicament forms in the form of injection solutions, drops or juices, as semi-solid medicament forms in the form of granules, tablets, pellets, patches, capsules, plasters/spray-on plasters or aerosols. The choice of auxiliary substances etc. and the amounts thereof to be used are dependent on whether the medicament is to be administered orally, perorally, parenterally, intravenously, intraperitoneally, intradermally, intramuscularly, intranasally, buccally, rectally or locally, for example to the skin, the mucosa or into the eyes. Preparations in the form of tablets, dragées, capsules, granules, drops, juices and syrups are suitable for oral administration, and solutions, suspensions, readily reconstitutable dry preparations and sprays are suitable for parenteral, topical and inhalatory administration. Compounds according to the invention in a depot, in dissolved form or in a plaster, optionally with the addition of agents that promote penetration through the skin, are suitable percutaneous forms of administration. Forms of preparation for administration orally or percutaneously can release the compounds according to the invention in a delayed manner. The compounds according to the invention can also be administered in parenteral long-term depot forms such as, for example, implants or implanted pumps. In principle, other further active ingredients known to the person skilled in the art can be added to the medicaments according to the invention.

The medicaments according to the invention are suitable for influencing KCNQ2/3 channels and exert an agonistic or antagonistic action, in particular an agonistic action.

The medicaments according to the invention are preferably suitable for the treatment of disorders or diseases that are mediated at least in part by KCNQ2/3 channels.

The medicaments according to the invention are suitable preferably for the treatment of one or more diseases selected from the group consisting of pain, especially pain selected from the group consisting of acute pain, chronic pain, neuropathic pain, muscular pain and inflammatory pain; epilepsy, urinary incontinence, anxiety, dependency, mania, bipolar disorders, migraine, cognitive diseases, dystonia-associated dyskinesias and/or urinary incontinence.

The medicaments according to the invention are suitable particularly preferably for the treatment of pain, most particularly preferably of chronic pain, neuropathic pain, inflammatory pain and muscular pain.

The medicaments according to the invention are also particularly preferably suitable for the treatment of epilepsy.

The invention further provides the use of at least one substituted 3-aminoisoxazolo-pyridine according to the invention, and optionally one or more pharmaceutically acceptable auxiliary substances, in the preparation of a medicament for the treatment of disorders or diseases that are mediated at least in part by KCNQ2/3 channels.

Preference is given to the use of at least one substituted 3-aminoisoxazolopyridine according to the invention, and optionally one or more pharmaceutically acceptable auxiliary substances, in the preparation of a medicament for the treatment of pain, especially pain selected from the group consisting of acute pain, chronic pain, neuropathic pain, muscular pain and inflammatory pain; epilepsy, urinary incontinence, anxiety, dependency, mania, bipolar disorders, migraine, cognitive diseases, dystonia-associated dyskinesias and/or urinary incontinence.

Particular preference is given to the use of at least one substituted 3-aminoisoxazolo-pyridine according to the invention, and optionally one or more pharmaceutically acceptable auxiliary substances, in the preparation of a medicament for the treatment of pain, most particularly preferably chronic pain, neuropathic pain, inflammatory pain and muscular pain.

Particular preference is given also to the use of at least one substituted 3-amino-isoxazolopyridine according to the invention, and optionally one or more pharmaceutically acceptable auxiliary substances, in the preparation of a medicament for the treatment of epilepsy.

The invention further provides at least one substituted 3-aminoisoxazolopyridine according to the invention, and optionally one or more pharmaceutically acceptable auxiliary substances, for the treatment of disorders or diseases that are mediated at least in part by KCNQ2/3 channels.

The invention further provides at least one substituted 3-aminoisoxazolopyridine according to the invention, and optionally one or more pharmaceutically acceptable auxiliary substances, for the treatment of pain, especially pain selected from the group consisting of acute pain, chronic pain, neuropathic pain, muscular pain and inflammatory pain; epilepsy, urinary incontinence, anxiety, dependency, mania, bipolar disorders, migraine, cognitive diseases, dystonia-associated dyskinesias and/or urinary incontinence.

Particular preference is given to at least one substituted 3-aminoisoxazolopyridine according to the invention, and optionally one or more pharmaceutically acceptable auxiliary substances, for the treatment of pain, most particularly preferably of chronic pain, neuropathic pain, inflammatory pain and muscular pain.

Particular preference is given also to at least one substituted 3-aminoisoxazolo-pyridine according to the invention, and optionally one or more pharmaceutically acceptable auxiliary substances, for the treatment of epilepsy.

The effectiveness against pain can be shown, for example, in the Bennett or Chung model (Bennett, G. J. and Xie, Y. K., A peripheral mononeuropathy in rat that produces disorders of pain sensation like those seen in man, Pain 1988, 33(1), 87-107; Kim, S. H. and Chung, J. M., An experimental model for peripheral neuropathy produced by segmental spinal nerve ligation in the rat, Pain 1992, 50(3), 355-363). The effectiveness against epilepsy can be demonstrated, for example, in the DBA/2 mouse model (De Sarro et al., Naunyn-Schmiedeberg's Arch. Pharmacol. 2001, 363, 330-336).

The substituted 3-aminoisoxazolopyridines according to the invention preferably have an EC₅₀ value of not more than 10 μM or not more than 5 μM, more preferably not more than 3 μM or not more than 2 μM, yet more preferably not more than 1.7 μM or not more than 1 μM, most preferably not more than 0.9 μM or not more than 0.6 μM and especially not more than 0.5 μM or not more than 0.3 μM. Methods for determining the EC₅₀ value are known to the person skilled in the art. The EC₅₀ value is preferably determined by fluorimetry, particularly preferably as described under “Pharmacological Experiments”.

The invention further provides processes for the preparation of the substituted 3-aminoisoxazolopyridines according to the invention.

The chemicals and reaction components used in the reactions described hereinbelow are available commercially or can in each case be prepared by conventional methods known to the person skilled in the art.

### General Preparation Processes

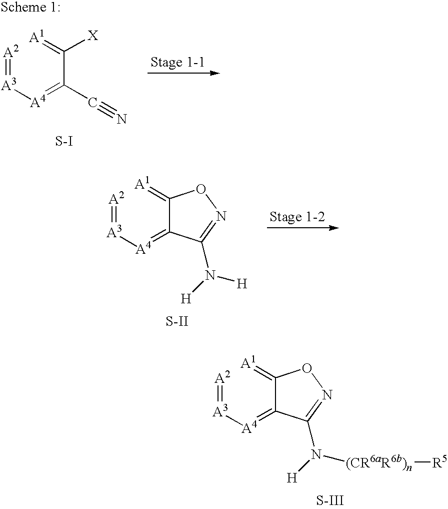

In stage 1-1, the aromatic ortho-halo-substituted nitrile (X=halogen, preferably F or Cl) S—I can be converted into the bicyclic compound S-II by means of a method known to the person skilled in the art, for example reaction with acethydroxamic acid with ring closure, optionally in the presence of a base.

The primary amine S-II can be converted into compound S-III in stage 1-2 by means of methods known to the person skilled in the art, for example an alkylation using the alkyl halide R⁵—(CR^{6a}R^{6b})ₙ-Hal or a reductive amination using the corresonding carbonyl compound.

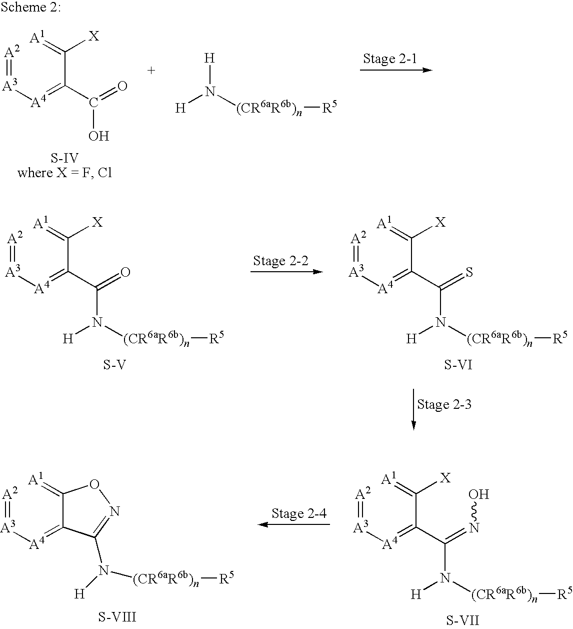

In stage 2-I, the carboxylic acid S-IV can be converted into the corresponding amide S-V by means of methods known to the person skilled in the art. For example, S-IV can first be converted by means of a suitable chlorinating agent, such as thionyl chloride, into the acid chloride, which is subsequently converted into the amide S-V using the primary amine R⁵—(CR^{6a}R^{6b})ₙ—NH₂, optionally in the presence of a base.

In stage 2-2, the amide S-V can be converted into the corresponding thioamide S-VI by means of methods known to the person skilled in the art. This can be carried out, for example, by reaction with a thionating agent such as Lawesson's reagent.

In stage 2-3, the thioamide S-VI can be reacted with hydroxylamine according to a method known to the person skilled in the art, optionally in the presence of a catalyst, to give compound S-VII, which can be converted in stage 2-4 into the bicyclic compound S-VIII likewise according to a method known to the person skilled in the art, in the presence of an acid, with ring closure.

The methods known to the person skilled in the art for carrying out reaction steps 1-1 to 2-4 are to be found in standard literature of organic chemistry, for example J. March, Advanced Organic Chemistry, Wiley & Sons, 6th edition, 2007; F. A. Carey, R. J. Sundberg, Advanced Organic Chemistry, Parts A and B, Springer, 5th edition, 2007); team of authors, Compendium of Organic Synthetic Methods, Wiley & Sons. Further methods and literature references can additionally be issued by customary databases such as the Reaxys® database of Elsevier, Amsterdam, NL or the SciFinder® database of the American Chemical Society, Washington, USA.

### Description of the Exemplary Syntheses

### Abbreviations

AcOH acetic acid
aq. aqueous
brine sat. aq. NaCl soln.
d days
DBU 1,8-diazabicyclo[5.4.0]undec-7-ene
DCM dichloromethane

### DIPEA N,N-diisopropylethylamine

EA ethyl acetate
sat. saturated
h hour(s)
i-PrOH 2-propanol
soln. solution
m/z mass to charge ratio
MeCN acetonitrile
MeOH methanol
min minutes
MS mass spectrometry
N/A not available
NEt₃ triethylamine
RG retigabine
RT room temperature 23±7° C.
CC column chromatography on silica gel
TFAA trifluoroacetic acid anhydride
THF tetrahydrofuran
vv ratio by volume

All starting materials not described explicitly were either available commercially (suppliers can be found, for example, in the Symyx® Available Chemicals database of MDL, San Ramon, US) or their synthesis has already been described exactly in the specialist literature (experimental procedures can be found, for example, in the Reaxys® database of Elsevier, Amsterdam, NL) or can be prepared by methods known to the person skilled in the art.

Silica gel 60 (0.040-0.063 mm) was used as the stationary phase for column chromatography (CC).

The analytical characterization of all intermediates and exemplary compounds was carried out by means of ¹H-NMR spectroscopy. Investigations by mass spectrometry (MS, m/z indicated for [M+H]⁺) were additionally carried out for all exemplary compounds and chosen intermediates.

### SYNTHESIS OF THE INTERMEDIATES

### Synthesis of Intermediate VW001: 3-Fluoro-2-(trifluoromethyl)-isonicotinonitrile

### a) Synthesis of 3-fluoro-2-(trifluoromethyl)-isonicotinamide

3 drops of DMF were added to a solution of 2.0 g (9.6 mmol) of 3-fluoro-2-(trifluoro-methyl)-isonicotinic acid in thionyl chloride (15 ml), and the mixture was heated for 3 h at 80° C. After cooling, concentration was carried out in vacuo. A saturated solution of NH₃ in THF (15 ml) at −78° C. was added to the resulting residue. The reaction mixture was slowly heated to RT and stirred for a further 30 min. The insoluble residue was filtered off and then washed with THF. The resulting crude product 3-fluoro-2-(trifluoromethyl)-isonicotinamide was reacted further without additional purification.

### b) Synthesis of 3-fluoro-2-(trifluoromethyl)-isonicotinonitrile

4.5 ml (33.6 mmol) of NEt₃ were added to a solution of the crude product described under VW001a) in DCM (40 ml), and the mixture was cooled to 0° C. 2.0 ml (14.4 mmol) of TFAA were then added, and stirring was carried out for 30 min at 0° C. and for a further 60 min at RT. The mixture was then poured onto ice and extracted with DCM. The organic phase was dried over MgSO₄, filtered and concentrated in vacuo. CC (hexane/EA 9:1) of the residue yielded 1.12 g (7.2 mmol, 75% over 2 stages) of 3-fluoro-2-(trifluoromethyl)-isonicotinonitrile.

### Synthesis of intermediate VX001: Isoxazolo[5,4-b]pyridin-3-amine

909 mg (8.1 mmol) of KO-t-Bu were added, with vigorous stirring, to a suspension of 668 mg (8.9 mmol) of acethydroxamic acid in DMF (20 ml), and the mixture was stirred for 30 min at RT. 990 mg (8.1 mmol) of 2-fluoro-nicotinonitrile were then added, and stirring was carried out for a further 5 h at 50° C. The mixture was then extracted with EA and the organic phase was dried over Na₂SO₄, filtered and concentrated in vacuo. CC (hexane/EA 7:3) of the residue yielded 305 mg (2.3 mmol, 28%) of isoxazolo[5,4-b]pyridin-3-amine.

### Synthesis of intermediate VX003: Isoxazolo[4,5-c]pyridin-3-amine

According to the process described for intermediate VX001, 143 mg (1.1 mmol, 13%) of isoxazolo[4,5-c]pyridin-3-amine were obtained from 1.12 g (8.1 mmol) of 4-chloro-nicotinonitrile.

### Synthesis of intermediate VX007: Isoxazolo[5,4-b]quinolin-3-amine

2.52 g (22.5 mmol) of KO-t-Bu were added to a solution of 1.69 g (22.5 mmol) of acethydroxamic acid in THF (45 ml), and stirring was carried out for 30 min at RT. The mixture was then cooled to 0° C., and 2.85 g (15.0 mmol) of 2-chloroquinoline-3-carbonitrile and 1.22 g (3.8 mmol) of Cs₂CO₃ were added in succession. The reaction solution was stirred for 3 h at RT and then diluted with EA, and washing with water and brine was carried out. The organic phase was dried over Na₂SO₄, filtered and concentrated in vacuo. CC (DCM/MeOH 97:3) of the residue yielded 700 mg (3.8 mmol, 25%) of isoxazolo[5,4-b]quinolin-3-amine.

### Synthesis of intermediate VY001: 1-(1-Bromoethyl)-3,5-dimethylbenzene

### a) Synthesis of 1-(3,5-dimethylphenyl)ethanol

1.02 g (27.0 mmol) of NaBH₄ were added at 0° C. to a solution of 2.0 g (13.5 mmol) of 3′,5′-dimethylacetophenone in an isopropanol/MeOH mixture (3:1 vv, 30 ml), and stirring was then carried out for 2 h at RT. Ice was then added, and concentration was carried out in vacuo. The residue was taken up in EA and washed with water and brine. The organic phase was dried over Na₂SO₄, filtered and concentrated in vacuo. 1.91 g (12.7 mmol, 94%) of 1-(3,5-dimethylphenyl)ethanol were obtained as residue.

### b) Synthesis of 1-(1-bromoethyl)-3,5-dimethylbenzene

1.3 ml (13.9 mmol) of PBr₃ were added dropwise, while cooling with ice, to a solution of 1.90 g (12.6 mmol) of 1-(3,5-dimethylphenyl)ethanol in toluene (30 ml). Stirring was then carried out for 2 h at RT. The mixture was then diluted with EA and neutralized with a sat. aq. Na₂CO₃ soln. The phases were separated, and the organic phase was washed with water and brine and dried over Na₂SO₄, filtered and concentrated in vacuo. CC (hexane) of the residue yielded 862 mg (4.0 mmol, 32%) of 1-(1-bromoethyl)-3,5-dimethyl-benzene.

### Synthesis of Further Intermediates

The synthesis of further intermediates was carried out according to the processes already described. Table 1 shows which compound was prepared by which process. It will be clear to the person skilled in the art which starting materials and reagents were used in each case.

TABLE 1

| | | Preparation | |
|---|---|---|---|
| Inter- | | analogous to | Yield |
| mediate | Chemical name | intermediate | [%] |
| VW002 | 2-chloro-4-fluoro-nicotinonitrile | VW001 | 57 |
| | | | (2 stages) |
| VX002 | isoxazolo[5,4-c]pyridin-3-amine | VX001 | 25 |
| VX004 | isoxazolo[4,5-b]pyridin-3-amine | VX001 | 46 |
| VX005 | 6-(trifluoromethyl)isoxazolo[5,4-b]- | VX003 | 20 |
| | pyridin-3-amine | | |
| VX006 | 6-methylisoxazolo[5,4-b]pyridin-3- | VX001 | 45 |
| | amine | | |
| VX008 | 7-(trifluoromethyl)isoxazolo[5,4-c]- | VX001 | 52 |
| | pyridin-3-amine | | |
| VX009 | 4-chloroisoxazolo[4,5-c]pyridin-3- | VX003 | 15 |
| | amine | | |
| VY002 | (4-(1-bromoethyl)phenyl)(trifluoro- | VY001 | 31 |
| | methyl)-sulfane | | (2 stages) |

### Synthesis of the Exemplary Compounds

### Synthesis of exemplary compound 2: N-(4-(Trifluoromethylthio)benzyl)-isoxazolo[5,4-b]pyridin-3-amine

454 mg (2.2 mmol) of 4-(trifluoromethylthio)benzaldehyde were added to a solution of 270 mg (2.0 mmol) of isoxazolo[5,4-b]pyridin-3-amine (intermediate VX001) in DCM (20 ml), and the mixture was stirred for 1 h at RT. 958 μl (6.0 mmol) of triethylsilane and 446 μl (6.0 mmol) of TFA were then added, and the reaction solution was stirred for 3 d at 60° C. After cooling to RT, the mixture was rendered basic with a sat. aq. NaHCO₃ soln. The phases were separated, and the aqueous phase was again extracted with DCM. The combined organic phases were dried over Na₂SO₄, filtered and concentrated in vacuo. CC (EA/hexane 7:3) of the residue yielded 373 mg (1.1 mmol, 58%) of N-(4-trifluoromethylthio)benzypisoxazolo[5,4-b]pyridin-3-amine. MS: m/z 326.0 [M+H]⁺.

### Synthesis of exemplary compound 5: N-(1-(4-(Trifluoromethylthio)phenyl)-ethypisoxazolo[5,4-b]pyridin-3-amine

484 mg (2.2 mmol) of 4′-(trifluoromethylthio)acetophenone were added to a solution of 270 mg (2.0 mmol) of isoxazolo[5,4-b]pyridin-3-amine (intermediate VX001) in DCM (20 ml), and the mixture was stirred for 1 h at RT. 958 μl (6.0 mmol) of triethylsilane and 446 μl (6.0 mmol) of TFA were then added, and the reaction solution was stirred for 7 d at 60° C. After cooling to RT, the mixture was rendered basic with a sat. aq. NaHCO₃ soln. The phases were separated, and the aqueous phase was again extracted with DCM. The combined organic phases were dried over Na₂SO₄, filtered and concentrated in vacuo. CC (EA/hexane 1:1) of the residue yielded 328 mg (1.0 mmol, 48%) of N-(1-(4-(trifluoromethylthio)phenyl)ethyl)-isoxazolo[5,4-b]pyridin-3-amine. MS: m/z 340.1 [M+H]⁺.

### Synthesis of exemplary compound 17: N-(1-(4-(Trifluoromethylthio)phenyl)-ethyl)-isoxazolo[4,5-c]pyridin-3-amine

58 mg (2.4 mmol) of NaH were added to a solution of 270 mg (2.0 mmol) of isoxazolo[4,5-c]pyridin-3-amine (intermediate VX003) in DMF (6 ml), and the mixture was cooled to 0° C. 684 mg (2.4 mmol) of (4-(1-bromoethyl)phenyl)(trifluoromethyl)-sulfane (intermediate VY002) were added at that temperature. Stirring was then carried out for a further 2 h at RT. A sat. aq. NH₄Cl soln. was then added, and extraction with EA was carried out. The organic phase was dried over Na₂SO₄, filtered and concentrated in vacuo. CC (EA/hexane 1:1) of the residue yielded 359 mg (1.1 mmol, 53%) of N-(1-(4-(trifluoromethylthio)phenyl)ethyl)-isoxazolo[4,5-c]-pyridin-3-amine. MS: m/z 340.1 [M+H]⁺.

### Synthesis of exemplary compound 26: 4-Pentylthio-N-(4-(trifluoromethylthio)-benzypisoxazolo[4,5-c]pyridin-3-amine

4.5 ml (30 mmol) of DBU were added to a solution of 3.7 ml (30 mmol) of 1-pentane-thiol in DMF (30 ml), and the mixture was then stirred for 1 h at 50° C. 1.08 g (3.0 mmol) of 4-chloro-N-(4-(trifluoromethylthio)benzyl)-isoxazolo[4,5-c]pyridin-3-amine (exemplary compound BSP025) were added at that temperature, and stirring was carried out for a further 16 h at 50° C. The mixture was then diluted with EA and washed with water and brine. The organic phase was dried over Na₂SO₄, filtered and concentrated in vacuo. CC (hexane/EA 9:1) of the residue yielded 103 mg (0.24 mmol, 8%) of 4-pentylthio-N-(4-(trifluoromethylthio)-benzyl)isoxazolo[4,5-c]-pyridin-3-amine. MS: m/z 428.1 [m+H]⁺.

### Synthesis of exemplary compound 27: N-(6-Chloropyridin-3-yl)isoxazolo[5,4-b]pyridin-3-amine

### a) Synthesis of N-(6-chloropyridin-3-yl)-2-fluoronicotinamide

A solution of 1.41 g (10.0 mmol) of 2-fluoro-nicotinic acid in thionyl chloride (60 ml) was heated for 2 h at 80° C. The mixture was then concentrated in vacuo, and excess thionyl chloride was removed by azeotropic distillation with toluene. The residue was taken up in dioxane (60 ml), and a solution of 1.29 g (10.0 mmol) of 3-amino-6-chloro-pyridine in dioxane (30 ml) was added. The reaction solution was heated for 90 min at 100° C. The mixture was then concentrated in vacuo, and the residue was washed with hexane. The resulting N-(6-chloropyridin-3-yl)-2-fluoronicotinamide (1.79 g, 7.1 mmol, 71%) was reacted further without additional purification.

### b) Synthesis of N-(6-chloropyridin-3-yl)-2-fluoropyridin-3-carbothioamide

8.09 g (20.0 mmol) of Lawesson's reagent were added at RT to a solution of 1.26 g (5.0 mmol) of N-(6-chloropyridin-3-yl)-2-fluoronicotinamide in a pyridine/toluene mixture (9:1 vv, 18 ml), and the mixture was then heated for 6 h at 110° C. After cooling to RT, quenching was carried out with a sat. aq. NaHCO₃ soln. The mixture was then extracted with EA, and the organic phase was washed with brine, dried over MgSO₄, filtered and concentrated in vacuo. CC (hexane/EA 8:1) of the residue yielded 534 mg (2.0 mmol, 40%) of N-(6-chloropyridin-3-yl)-2-fluoropyridin-3-carbo-thioamide.

### c) Synthesis of N-(6-chloropyridin-3-yl)-2-fluoro-N′-hydroxynicotinimideamide

139 mg (4.0 mmol) of hydroxylamine hydrochloride and 656 mg (8.0 mmol) of sodium acetate were added to a solution of 534 mg (2.0 mmol) of N-(6-chloropyridin-3-yl)-2-fluoropyridin-3-carbothioamide in EtOH (10 ml), and the mixture was then heated for 4 h at 80° C. Insoluble solids were filtered off, and the filtrate was concentrated in vacuo. The residue was taken up in EA, and the solution was washed with water and brine, dried over Na₂SO₄, filtered and concentrated in vacuo. The residue was washed with an EA/hexane mixture (9:1 vv), yielding 317 mg (1.2 mmol, 60%) of N-(6-chloropyridin-3-yl)-2-fluoro-N′-hydroxynicotinimideamide.

### d) Synthesis of N-(6-chloropyridin-3-yl)isoxazolol[5,4-b]pyridin-3-amine

415 mg (3.0 mmol) of potassium carbonate were added to a solution of 267 mg (1.0 mmol) of N-(6-chloropyridin-3-yl)-2-fluoro-N′-hydroxynicotinimideamide in DMF (5 ml), and the mixture was heated for 16 h at 70° C. The mixture was then diluted with water and extracted with EA. The organic phase was washed with water and brine, dried over Na₂SO₄, filtered and concentrated in vacuo. CC (hexane/EA 9:1) of the residue yielded 187 mg (0.8 mmol, 76%) of N-(6-chloropyridin-3-yl)isoxazolo-[5,4-b]pyridin-3-amine.

### Synthesis of Further Exemplary Compounds

The synthesis of further exemplary compounds was carried out according to the processes already described. Table 2 shows which compound was prepared by which process. It will be clear to the person skilled in the art which starting materials and reagents were used in each case.

TABLE 2

| | | Preparation | | |
|---|---|---|---|---|
| | | analogous to | Yield | MS m/z |
| Example | Chemical name | example | [%] | [M + H]⁺ |
| 1 | N-(1-(4-(trifluoromethyl)phenyl)- | 5 | 27 | 308.1 |
| | ethyl)-isoxazolo[5,4-b]pyridin-3- | | | |
| | amine | | | |
| 3 | N-(3,5-dimethyl-benzyl)isoxazolo- | 2 | 55 | 254.1 |
| | [5,4-b]pyridin-3-amine | | | |
| 4 | N-(1-(3,5-dimethyl-phenyl)ethyl)- | 5 | 49 | 268.1 |
| | isoxazolo[5,4-b]pyridin-3-amine | | | |
| 6 | N-(4-(trifluoromethylthio)benzyl)- | 2 | 40 | 326.0 |
| | isoxazolo[5,4-c]pyridin-3-amine | | | |
| 7 | N-(3,5-dimethyl-benzyl)isoxazolo- | 2 | 71 | 254.1 |
| | [5,4-c]pyridin-3-amine | | | |
| 8 | N-(3,5-dimethyl-benzyl)isoxazolo- | 2 | 69 | 254.1 |
| | [4,5-b]pyridin-3-amine | | | |
| 9 | N-(4-(trifluoromethylthio)benzyl)- | 2 | 75 | 326.0 |
| | isoxazolo[4,5-b]pyridin-3-amine | | | |
| 10 | N-(1-(3,5-dimethyl-phenyl)ethyl)- | 5 | 63 | 268.1 |
| | isoxazolo[4,5-b]pyridin-3-amine | | | |
| 11 | N-(3,5-dimethyl-benzyl)isoxazolo- | 2 | 62 | 254.1 |
| | [4,5-c]pyridin-3-amine | | | |
| 12 | N-(4-(trifluoromethylthio)benzyl)- | 2 | 68 | 326.0 |
| | isoxazolo[4,5-c]pyridin-3-amine | | | |
| 13 | N-(1-(4-(trifluoromethylthio)phenyl)- | 5 | 64 | 340.1 |
| | ethyl)isoxazolo[4,5-b]pyridin-3- | | | |
| | amine | | | |
| 14 | N-(1-(4-(trifluoromethyl)phenyl)- | 17 | 32 | 340.1 |
| | ethyl)isoxazolo[5,4-c]pyridin-3- | | | |
| | amine | | | |
| 15 | N-(1-(3,5-dimethyl-phenyl)ethyl)- | 17 | 34 | 268.1 |
| | isoxazolo[5,4-c]pyridin-3-amine | | | |
| 16 | N-(1-(3,5-dimethyl-phenyl)ethyl)- | 17 | 58 | 268.1 |
| | isoxazolo[4,5-c]pyridin-3-amine | | | |
| 18 | N-(1-(3,5-dimethylphenyl)ethyl)-6- | 17 | 48 | 336.1 |
| | (trifluoromethyl)isoxazolo[5,4-b]- | | | |
| | pyridin-3-amine | | | |
| 19 | N-(4-(trifluoromethylthio)benzyl)-6- | 2 | 59 | 394.0 |
| | trifluoromethyl-isoxazolo[5,4-b]- | | | |
| | pyridin-3-amine | | | |
| 20 | 6-methyl-N-(4-(trifluoromethylthio)- | 2 | 95 | 340.1 |
| | benzyl)-isoxazolo[5,4-b]pyridin-3- | | | |
| | amine | | | |
| 21 | N-(1-(3,5-dimethyl-phenyl)ethyl)-7- | 17 | 60 | 336.1 |
| | trifluoromethyl-isoxazolo[5,4-c]- | | | |
| | pyridin-3-amine | | | |
| 22 | N-(1-(3,5-dimethylphenyl)ethyl)-6- | 17 | 63 | 282.2 |
| | methyl-isoxazolo[5,4-b]pyridin-3- | | | |
| | amine | | | |
| 23 | N-(4-(trifluoromethylthio)benzyl)-7- | 2 | 75 | 394.0 |
| | trifluoromethyl-isoxazolo[5,4-c]- | | | |
| | pyridin-3-amine | | | |
| 24 | N-(4-(trifluoromethylthio)benzyl)- | 2 | 60 | 376.1 |
| | isoxazolo[5,4-b]quinolin-3-amine | | | |
| 25 | 4-chloro-N-(4-(trifluoromethylthio)- | 2 | 60 | 360.0 |
| | benzyl)-isoxazolo[4,5-c]pyridin-3- | | | |
| | amine | | | |
| 28 | N-(6-chloropyridin-3-yl)-6- | 27 | 37 | 315.0 |
| | (trifluoromethyl)isoxazolo[5,4-b]- | | (4 stages) | |
| | pyridin-3-amine | | | |
| 29 | N-(6-chloropyridin-3-yl)isoxazolo- | 27 | 24 | 247.0 |
| | [5,4-c]pyridin-3-amine | | (4 stages) | |
| 30 | N-(6-chloropyridin-3-yl)-7- | 27 | 12 | 315.0 |
| | (trifluoromethyl)isoxazolo[5,4-c]- | | (4 stages) | |
| | pyridin-3-amine | | | |
| 31 | N-phenyl-6-(trifluoromethyl)- | 27 | 29 | 280.1 |
| | isoxazolo[5,4-b]pyridin-3-amine | | (4 stages) | |
| 32 | N-phenyl-7- | 27 | 17 | 280.1 |
| | (trifluoromethyl)isoxazolo[5,4-c]- | | (4 stages) | |
| | pyridin-3-amine | | | |

### Pharmacological Experiments

### Fluorescence Assay Using a Voltage Sensitive Dye

Human CHO-K1 cells expressing KCNQ2/3 channels are cultivated adherently at 37° C., 5% CO₂ and 95% humidity in cell culture bottles (e.g. 80 cm² TC flasks, Nunc) with DMEM-high glucose (Sigma Aldrich, D7777) including 10% FCS (PAN Biotech, e.g. 3302-P270521) or alternatively MEM Alpha Medium (1×, liquid, Invitrogen, #22571), 10% fetal calf serum (FCS) (Invitrogen, #10270-106, heat-inactivated) and the necessary selection antibiotics.

Before being sown out for the measurements, the cells are washed with a 1×DPBS buffer without Ca²⁺/Mg²⁺ (e.g. Invitrogen, #14190-094) and detached from the bottom of the culture vessel by means of Accutase (PAA Laboratories, #L11-007) (incubation with Accutase for 15 min at 37° C.). The cell count then present is determined using a CASY™ cell counter (TCC model, Scharfe System) in order subsequently to apply, depending on the density optimization for the individual cell line, 20,000-30,000 cells/well/100 μl of the described nutrient medium to 96-well measuring plates of the Corning™ CelIBIND™ type (Flat Clear Bottom Black Polystyrene Microplates, #3340). Incubation is then carried out for one hour at room temperature, without gassing or adjusting the humidity, followed by incubation for 24 hours at 37° C., 5% CO₂ and 95% humidity.

The voltage-sensitive fluorescent dye from the Membrane Potential Assay Kit (Red™ Bulk format part R8123 for FLIPR, MDS Analytical Technologies™) is prepared by dissolving the contents of a vessel Membrane Potential Assay Kit Red Component A in 200 ml of extracellular buffer (ES buffer, 120 mM NaCl, 1 mM KCl, 10 mM HEPES, 2 mM CaCl₂, 2 mM MgCl₂, 10 mM glucose; pH 7.4). After removal of the nutrient medium, the cells are washed with 200 μl of ES buffer, then covered with a layer of 100 μl of the dye solution prepared above and incubated for 45 min at room temperature with the exclusion of light.

The fluorescence measurements are carried out with a BMG Labtech FLUOstar™ BMG Labtech NOVOstar™ or BMG Labtech POLARstar™ instrument (525 nm excitation, 560 nm emission, Bottom Read mode). After incubation of the dye, 50 μl of the test substances in the desired concentrations, or 50 μl of ES buffer for control purposes, are introduced into separate cavities of the measuring plate and incubated for 30 min at room temperature while being shielded from light. The fluorescence intensity of the dye is then measured for 5 min and the fluorescence value F₁ of each well is thus determined at a given, constant time. 15 ml of a 100 mM KCl solution (final concentration 92 mM) are then added to each well. The change in fluorescence is subsequently measured until all the relevant measured values have been obtained (mainly 5-30 min). At a given time after KCl application, a fluorescence value F₂ is determined, in this case at the time of the fluorescence peak.

For calculation, the fluorescence intensity F₂ is compared with the fluorescence intensity F₁, and the agonistic activity of the target compound on the potassium channel is determined therefrom. F₂ and F₁ are calculated as follows:

$$( \frac{{F}_{2} - {F}_{1}}{{F}_{1}} ) \times 100 = \frac{Δ F}{F} ( \% )$$

In order to determine whether a substance has agonistic activity,

$$\frac{Δ F}{F} ,$$

for example, can be compared with

$${( \frac{Δ F}{F} )}_{K}$$

of control cells.

$${( \frac{Δ F}{F} )}_{K}$$

is determined by adding to the reaction batch only the buffer solution instead of the test substance, determining the value F_{1K} of the fluorescence intensity, adding the potassium ions as described above, and measuring a value F_{2K} of the fluorescence intensity. F_{2K} and F_{1K} are then calculated as follows:

$$( \frac{{F}_{2 K} - {F}_{1 K}}{{F}_{1 K}} ) \times 100 = {( \frac{Δ F}{F} )}_{K} ( \% )$$

A substance has an agonistic activity on the potassium channel when

$$\frac{Δ F}{F}$$

is greater than

$${( \frac{Δ F}{F} )}_{K} \text{:}$$

$$\frac{Δ F}{F} > {( \frac{Δ F}{F} )}_{K}$$

Independently of the comparison of

$$\frac{Δ F}{F}$$

with

$${( \frac{Δ F}{F} )}_{K}$$

it is possible to conclude that a target compound has agonistic activity if an increase in

$$\frac{Δ F}{F}$$

is to be observed as the dosage of the target compound increases.

Calculations of EC₅₀ values are carried out with the aid of ‘Prism v4.0’ software (GraphPad Software™).

### Pharmacological Data

The results from the pharmacological models described above are summarized in Table 3.

TABLE 3

| | Fluorimetry % | Fluorimetry |
|---|---|---|
| Exemplary | efficacy @ 10 μM | EC₅₀ |
| compound | (retigabine = 100%) | [nM] |
| 1 | 43 | |
| 2 | 77 | 1695 |
| 3 | 34 | |
| 4 | 65 | |
| 5 | 97 | |
| 6 | 65 | |
| 7 | 29 | |
| 9 | 86 | |
| 10 | 27 | |
| 12 | 39 | |
| 13 | 50 | |
| 14 | 78 | |
| 15 | 73 | |
| 16 | 15 | |
| 17 | 49 | 899 |
| 18 | 43 | |
| 19 | 41 | |
| 20 | 49 | |
| 21 | 39 | |
| 22 | 49 | 231 |
| 23 | 27 | |
| 24 | 38 | 411 |
| 25 | 19 | |

### Comparison Experiments

The substituted 3-aminoisoxazolopyridines according to the invention are distinguished by improved solubility in aqueous media, which may lead inter alia to improved oral bioavailability.

To that end, the intrinsic solubility S was first calculated for a group of chosen comparison examples A1-A3, B1-B3, C1, C2, D1 and D2 (software: Advanced Chemistry Development ACD/PhysChem, ACD/Labs Release/Version 10.00 Build 13903, 3 Oct. 2006). The phenyl group of the benzyl substituent of examples A1-A3 was replaced by a pyridyl group in each of examples B1-B3, C1, C2, D1 and D2. It was found, surprisingly, that the introduction of a nitrogen atom into the systems in question can lead to an increase in solubility by a factor of about 10 (see Table 4):

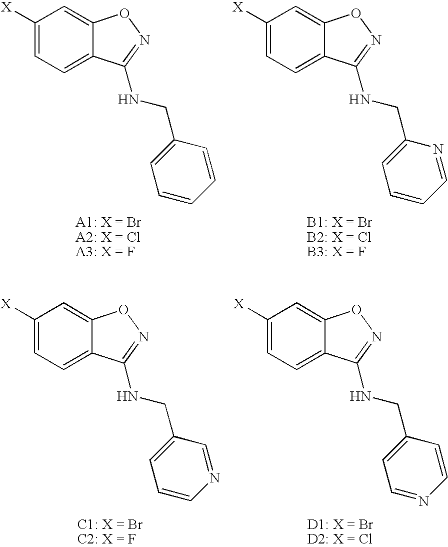

However, it will be seen from Table 4 that, although the solubility in aqueous media is improved as compared with compounds A1-A3, the pharmacological activity in respect of the KCNQ2/3 receptor is not retained at the same time.

For that reason attempts were made to introduce a nitrogen atom into the phenyl ring of the benzoisoxazole structure instead, and in that manner the exemplary compounds 2, 6, 9 and 12 inter alia were found, the activity of which towards the KCNQ2/3 receptor is, surprisingly, still present. In comparison with compound E1, exemplary compounds 2, 6, 9 and 12 additionally exhibit a solubility increased by a factor of about 10.

TABLE 4

| | calculated | KCNQ2/3 |
|---|---|---|
| | solubility | stimulation |
| Substance | log S [μmol/l] | at 10 μM [%] |
| A1 | −3.7 | 23 |
| A2 | −3.77 | 58 |
| A3 | −3.24 | 70 |
| B1 | −2.65 | 4 |
| B2 | −2.71 | 0 |
| B3 | −2.18 | −4 |
| C1 | −2.7 | 5 |
| C2 | −2.24 | −3 |
| D1 | −2.71 | 0 |
| D2 | −2.77 | 1 |
| E1 | −5.11 | 75 |
| Example 2 | −4.1 | 77 |
| Example 6 | −4.06 | 65 |
| Example 9 | −4.1 | 86 |
| Example 12 | −4.06 | 39 |

E1

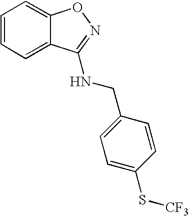

Example 2

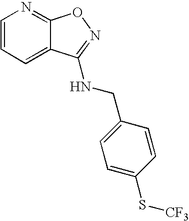

Example 6

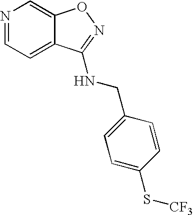

Example 12

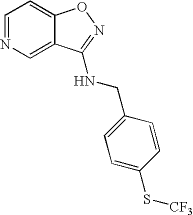

Example 9

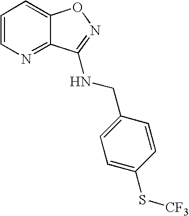

1. A substituted 3-aminoisoxazolopyridine of the general formula (1): 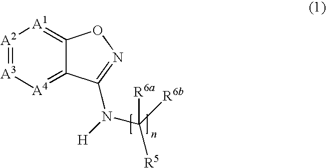
wherein
A¹ represents N or C—R¹,
A² represents N or C—R²,
A³ represents N or C—R³,
A⁴ represents N or C—R⁴,
wherein precisely one of A¹, A², A³ or A⁴ represents N;
R⁰ represents C₁₋₁₀-alkyl or C₂₋₁₀-heteroalkyl, in each case saturated or unsaturated, branched or unbranched, unsubstituted or mono- or poly-substituted; C₃₋₁₀-cycloalkyl or heterocyclyl, in each case saturated or unsaturated, unsubstituted or mono- or poly-substituted; aryl or heteroaryl, in each case unsubstituted or mono- or poly-substituted; C₁₋₈-alkyl- or C₂₋₈-heteroalkyl-bridged C₃₋₁₀-cycloalkyl or heterocyclyl, in each case saturated or unsaturated, unsubstituted or mono- or poly-substituted, wherein the alkyl chain or heteroalkyl chain in each case can be branched or unbranched, saturated or unsaturated, unsubstituted, mono- or poly-substituted; or C₁₋₈-alkyl- or C₂₋₈-heteroalkyl-bridged aryl or heteroaryl, in each case unsubstituted or mono- or poly-substituted, wherein the alkyl chain or heteroalkyl chain in each case can be branched or unbranched, saturated or unsaturated, unsubstituted, mono- or poly-substituted;
R¹, R², R³ and R⁴ each independently of the others denotes H; F; Cl; Br; I; NO₂; CF₃; CN; R⁰; C(═O)H; C(═O)R⁰; C(═O)OH; C(═O)OR⁰; C(═O)NH₂; C(═O)NHR⁰; C(═O)N(R⁰)₂; OH; OR⁰; O—C(═O)—R⁰; O—C(═O)—O—R⁰; O—(C═O)—NH—R⁰; O—C(═O)—N(R⁰)₂; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NO—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SR⁰; S(═O)R⁰; S(═O)₂R⁰; S(═O)₂OH; S(═O)₂OR⁰; S(═O)₂NH₂; S(═O)₂NHR⁰; or S(═O)₂N(R⁰)₂;
or R¹ and R²; or R² and R³; or R³ and R⁴, together with the carbon atoms joining them, form an aryl or heteroaryl, in each case unsubstituted or mono- or poly-substituted;
with the proviso that R¹ may not represent NH—R⁰ when A⁴ represents N;
R⁵ represents aryl or heteroaryl, in each case unsubstituted or mono- or poly-substituted; with the proviso that when R⁵ denotes heteroaryl, unsubstituted or mono- or poly-substituted, the bonding of the heteroaryl takes place via a carbon atom of the heteroaryl;
R^{6a}, R^{6b} independently of one another represent H or R⁰;
n represents a natural number in the range from 1 to 6;
wherein “substituted” in the case of substitution on alkyl, heteroalkyl, heterocyclyl and cycloalkyl stands for the substitution of one or more hydrogen atoms, in each case independently of one another, by F; Cl; Br; I; CN; CF₃; ═NH; ═C(NH₂)₂; NO₂; R⁰; C(═O)H; C(═O)R⁰; CO₂H; C(═O)OR⁰; CONH₂; C(═O)NHR⁰; C(═O)N(R⁰)₂; OH; OR⁰; —O—(C₁₋₈-alkyl)-O—; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SR⁰; S(═O)R⁰; S(═O)₂R⁰; S(═O)₂H; S(═O)₂OH; S(═O)₂OR⁰;
S(═O)₂NH₂; S(═O)₂NHR⁰; or S(═O)₂N(R)₂;
wherein “substituted” in the case of substitution on aryl and heteroaryl stands for the substitution of one or more hydrogen atoms, in each case independently of one another, by F; Cl; Br; I; NO₂; CF₃; CN; R⁰; C(═O)H; C(═O)R⁰; CO₂H; C(═O)OR⁰; CONH₂; C(═O)NHR⁰; C(═O)N(R⁰)₂; OH; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂, SH; SR⁰; S(═O)R⁰; S(═O)₂R⁰; S(═O)₂OH; S(═O)₂OR⁰; S(═O)₂NH₂;
S(═O)₂NHR⁰; or S(═O)₂N(R)₂;
with the exception of the following compound:
4-methoxy-N-(2-methyl-2-phenylpropyl)isoxazolo[5,4-c]pyridin-3-amine;
said substituted 3-aminoisoxazolopyridine being in the form of thea free compound or a salt of a physiologically acceptable acid or base.

2. The isoxazolopyridine as claimed in claim 1, wherein
R¹, R², R³ and R⁴ each independently of the others denotes H; F; Cl; Br; I; NO₂; CF₃; CN; R⁰; C(═O)(R⁰ or H); C(═O)O(R⁰ or H); C(═O)N(R⁰ or H)₂; OH; OR⁰; O—(C₁₋₈-alkyl)-OH; O—(C₁₋₈-alkyl)-O—C₁₋₈-alkyl; OCF₃; N(R⁰ or H)₂; N(R⁰ or H)—C(═O)—R⁰; N(R⁰ or H)—C(═O)—N(R⁰ or H)₂; SH; SCF₃; Se; S(═O)₂R⁰; S(═O)₂O(R⁰ or H); and S(═O)₂—N(R⁰ or H)₂;
or R¹ and R²; or R² and R³; or R³ and R⁴, together with the carbon atoms joining them, form an aryl or heteroaryl, in each case unsubstituted or mono- or poly-substituted;
with the proviso that R¹ may not represent NH—R⁰, C(═O)N(R⁰ or H)₂, N(R⁰ or H)—C(═O)—N(R⁰ or H)—C(═O)—N(R⁰ or H)₂ or S(═O)₂—N(R⁰ or H)₂ when A⁴ represents N.

3. The isoxazolopyridine as claimed in claim 1, wherein
R¹ and R² each independently of the other represents H; F; Cl; Br; I; NO₂; CN; OH; O—C₁₋₄-alkyl; O—(C₁₋₄-alkyl)-OH; O—(C₁₋₄-alkyl)-O—C₁₋₈-alkyl; OCF₃; C₁₋₄-alkyl; C(═O)—OH; CF₃; NH₂; NH(C₁₋₄-alkyl); N(C₁₋₈-alkyl)₂; SH; S—C₁₋₄-alkyl; SCF₃; S(═O)₂OH; benzyl, aryl or heteroaryl, in each case unsubstituted or mono- or poly-substituted by one or more substituents selected independently of one another from the group consisting of F, Cl, Br, I, CN, OH, O—C₁₋₄-alkyl, OCF₃, C₁₋₄-alkyl, CF₃, and SCF₃;
R³ and R⁴ each independently of the other represents H; F; Cl; Br; I; NO₂; CN; OH; O—C₁₋₄-alkyl; O—(C₁₋₈-alkyl)-OH; O—(C₁₋₄-alkyl)-O—C₁₋₄-alkyl; OCF₃; C₁₋₄-alkyl; C(═O)—OH; CF₃; NH₂; NH(C₁₋₈-alkyl); N(C₁₋₈-alkyl)₂; SH; S—C₁₋₄-alkyl; SCF₃; S(═O)₂OH; C(═O)NH₂; C(═O)NH(C₁₋₈-alkyl); C(═O)N(C₁₋₄-alkyl)₂; C(═O)NH(aryl); C(═O)N(aryl)₂; C(═O)NH(heteroaryl); C(═O)N(heteroaryl)₂; C(═O)N(C₁₋₄-alkyl)(aryl); C(═O)N(C₁₋₄-alkyl)(heteroaryl); C(═O)N(heteroaryl)(aryl); NH—C(═O)—C₁₋₄-alkyl; NH—C(═O)-aryl; NH—C(═O)-heteroaryl; N(C₁₋₄-alkyl)-C(═O)—C₁₋₄-alkyl; N(C₁₋₄-alkyl)-C(═O)-aryl; N(C₁₋₄-alkyl)-C(═O)-heteroaryl; N(aryl)-C(═O)—C₁₋₄-alkyl; N(aryl)-C(═O)-aryl; N(heteroaryl)-C(═O)-heteroaryl; NH—C₁₋₈-alkyl-heterocyclyl; NH—C₁₋₈-alkyl-cycloalkyl; NH—C₁₋₄-alkyl-aryl; NH—C₁₋₄-alkyl-heteroaryl; N(C₁₋₄-alkyl)-C₁₋₈-alkyl-heterocyclyl; N(C₁₋₄-alkyl)-C₁₋₄-alkyl-cycloalkyl; N(C₁₋₄-alkyl)-C₁₋₈-alkyl-aryl; N(C₁₋₈-alkyl)-C₁₋₄-alkyl-heteroaryl; NH—C(═O)C₁₋₄-alkyl-heterocyclyl; NH—C(═O)C₁₋₈-alkyl-cycloalkyl; NH—C(═O)C₁₋₄-alkyl-aryl; NH—C(═O)C₁₋₈-alkyl-heteroaryl; N(C₁₋₈-alkyl)-C(═O)C₁₋₈-alkyl-heterocyclyl; N(C₁₋₈-alkyl)-C(═O)C₁₋₄-alkyl-cycloalkyl; N(C₁₋₄-alkyl)-C(═O)C₁₋₄-alkyl-aryl; N(C₁₋₈-alkyl)-C(═O)—C₁₋₄-alkyl-heteroaryl; benzyl; wherein aryl or heteroaryl in each case can be unsubstituted or mono- or poly-substituted by one or more substituents selected independently of one another from the group consisting of F, Cl, Br, I, CN, OH, O—C₁₋₄-alkyl, OCF₃, C₁₋₄-alkyl, CF₃, and SCF₃; and wherein cycloalkyl or heterocyclyl in each case can be unsubstituted or mono- or poly-substituted by one or more substituents selected independently of one another from the group consisting of F, Cl, Br, I, CN, OH, O—C₁₋₈-alkyl, OCF₃, C₁₋₄-alkyl, CF₃, and SCF₃;
or R¹ and R²; or R² and R³; or R³ and R⁴, together with the carbon atoms joining them, form an aryl or heteroaryl, in each case unsubstituted or mono- or poly-substituted by one or more substituents selected independently of one another from the group consisting of F, Cl, Br, I, CN, OH, O—C₁₋₈-alkyl, OCF₃, C₁₋₄-alkyl, CF₃, and SCF₃.

4. The isoxazolopyridine as claimed in claim 1, wherein
R⁵ represents aryl or heteroaryl, in each case unsubstituted or mono- or poly-substituted by one or more substituents selected independently of one another from the group consisting of F, Cl, Br, I, NO₂, CN, OH, O—C₁₋₈-alkyl, OCF₃, C₁₋₈-alkyl, C(═O)—OH, CF₃, NH₂, NH(C₁₋₈-alkyl), N(C₁₋₈-alkyl)₂, SH, S—C₁₋₈-alkyl, SCF₃, S(═O)₂OH, benzyl, unsubstituted, and phenyl,
wherein aryl or heteroaryl in each case can optionally be fused with C₃₋₁₀-cycloalkyl or heterocyclyl, in each case saturated or unsaturated, unsubstituted or mono- or poly-substituted by one or more substituents selected independently of one another from the group consisting of F, Cl, Br, I, NO₂, CN, OH, O—C₁₋₈-alkyl, OCF₃, C₁₋₈-alkyl, C(═O)—OH, CF₃, NH₂, NH(C₁₋₈-alkyl), N(C₁₋₈-alkyl)₂, SH, S—C₁₋₈-alkyl, SCF₃, S(═O)₂OH, benzyl, unsubstituted, and phenyl, unsubstituted.

5. The isoxazolopyridine as claimed in claim 1, wherein
R^{6a}, R^{6b} independently of one another represent methyl or ethyl optionally substituted by one or more substituents selected from the group consisting of F; Cl; Br; I; CF₃; —O—CH₃, and CN;
n is 1;
R⁵ represents phenyl, pyridyl or thienyl, in each case unsubstituted or mono- or poly-substituted by one or more substituents selected independently of one another from the group consisting of F, Cl, Br, I, NO₂, CN, OH, O—C₁₋₈-alkyl, OCF₃, C₁₋₈-alkyl, C(═O)—OH, CF₃, NH₂, NH(C₁₋₈-alkyl), N(C₁₋₈-alkyl)₂, SH, SCF₃, and S(═O)₂OH;
wherein phenyl and pyridyl in each case can optionally be fused with C₃₋₁₀-cycloalkyl, saturated or unsaturated, unsubstituted or mono- or poly-substituted by one or more substituents selected independently of one another from the group consisting of F, Cl, Br, I, NO_{B}, CN, OH, O—C₁₋₈-alkyl, OCF₃, C₁₋₈-alkyl, C(═O)—OH, CF₃, NH₂, NH(C₁₋₈-alkyl), N(C₁₋₈-alkyl)₂, SH, SCF₃, S(═O)₂OH and benzyl, unsubstituted; or phenyl and pyridyl in each case can optionally be fused with heterocyclyl, saturated or unsaturated, in which one or two carbon atoms have been replaced by a heteroatom in each case selected independently of one another from the group consisting of O, N, NH and N(C₁₋₈-alkyl), in each case unsubstituted or mono- or poly-substituted by one or more substituents selected independently of one another from the group consisting of F, Cl, Br, I, NO₂, CN, OH, O—C₁₋₄-alkyl, OCF₃, C(═O)—OH, CF₃, NH₂, NH(C₁₋₈-alkyl), SH, SCF₃, S(═O)₂OH and benzyl, unsubstituted.

6. The isoxazolopyridine as claimed in claim 1, having the general formula (2): 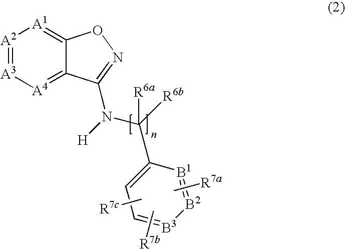
wherein
B¹ represents CH or N;
B² represents CH or N;
B³ represents CH or N;
wherein not more than one of B¹, B² and B³ can represent N;
R^{7a}, R^{7b} and R^{7c} each independently of the others represents H; F; Cl; Br; I; CN; OH; O—C₁₋₄-alkyl; OCF₃; C₁₋₈alkyl; CF₃; and SCF₃;
or the substituents R^{7a} and R^{7b}, together with the carbon atoms joining them, form one of the following partial structures I-a, I-b, I-c, I-d, I-e or I-f: 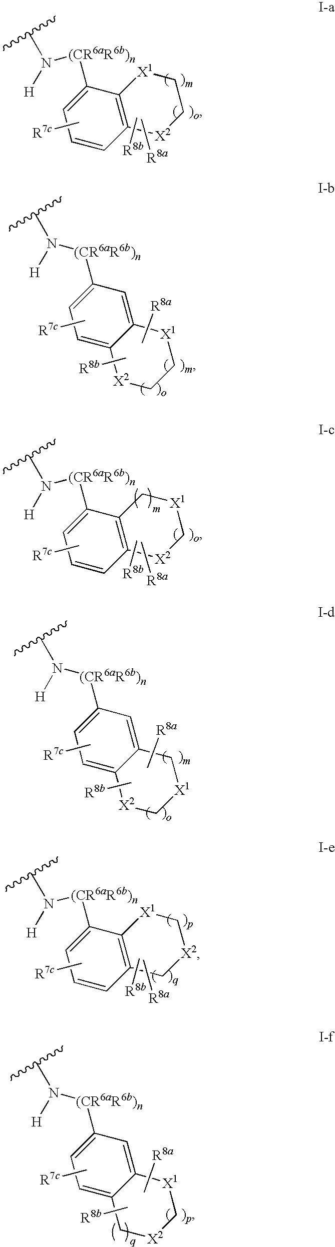
wherein
m in each case represents 0, 1 or 2;
o in each case represents 1, 2 or 3;
p in each case represents 1, 2 or 3;
q in each case represents 0, 1 or 2;
X¹ and X² each independently of the other represents CH₂, O, NH or N(C₁₋₄-alkyl);
R^{8a} and R^{8b} each independently of the other represents F, Cl, Br, I, NO₂, CN, OH, O—C₁₋₄-alkyl, OCF₃, C₁₋₄-alkyl, CF₃, NH₂, NH(C₁₋₈-alkyl) and N(C₁₋₄-alkyl)₂;
and the remaining substituents R^{6a}; and R^{6b} have the meanings given in claim 1; and R^{7c} has one of the meanings given above.

7. The isoxazolopyridine as claimed in claim 1, wherein
R¹, R², R³ and R⁴ each independently of the others denotes H; F; Cl; Br; I; CN; O—C₁₋₄-alkyl; OCF₃; C₁₋₈-alkyl; CF₃; NH₂; S—C₁₋₄-alkyl; or SCF₃;
or R¹ and R²; or R² and R³; or R³ and R⁴, together with the carbon atoms joining them, form an unsubstituted phenyl.

8. The isoxazolopyridine as claimed in claim 1, selected from the group consisting of:
1 N-(1-(4-(trifluoromethyl)phenyl)ethyl)-isoxazolo[5,4-b]pyridin-3-amine;
2 N-(4-(trifluoromethylthio)benzyl)isoxazolo-[5,4-b]pyridin-3-amine;
3 N-(3,5-dimethyl-benzyl)isoxazolo[5,4-b]pyridin-3-amine;
4 N-(1-(3,5-dimethyl-phenyl)ethyl)isoxazolo[5,4-b]pyridin-3-amine;
5 N-(1-(4-(trifluoromethylthio)phenyl)ethyl)-isoxazolo[5,4-b]pyridin-3-amine;
6 N-(4-(trifluoromethylthio)benzyl)isoxazolo[5,4-c]pyridin-3-amine;
7 N-(3,5-dimethyl-benzyl)isoxazolo[5,4-c]pyridin-3-amine;
8 N-(3,5-dimethyl-benzyl)isoxazolo[4,5-b]pyridin-3-amine;
9 N-(4-(trifluoromethylthio)benzypisoxazolo[4,5-b]pyridin-3-amine;
10 N-(1-(3,5-dimethyl-phenyl)ethyl)isoxazolo[4,5-b]pyridin-3-amine;
11 N-(3,5-dimethyl-benzyl)isoxazolo[4,5-c]pyridin-3-amine;
12 N-(4-(trifluoromethylthio)benzyl)isoxazolo[4,5-c]pyridin-3-amine;
13 N-(1-(4-(trifluoromethylthio)phenyl)ethyl)isoxazolo[4,5-b]pyridin-3-amine;
14 N-(1-(4-(trifluoromethyl)phenyl)ethyl)isoxazolo[5,4-c]pyridin-3-amine;
15 N-(1-(3,5-dimethyl-phenyl)ethyl)isoxazolo[5,4-c]pyridin-3-amine;
16 N-(1-(3,5-dimethyl-phenyl)ethypisoxazolo[4,5-c]pyridin-3-amine;
17 N-(1-(4-(trifluoromethylthio)phenyl)ethyp-isoxazolo[4,5-c]pyridin-3-amine;
18 N-(1-(3,5-dimethylphenyl)ethyl)-6-(trifluoromethypisoxazolo[5,4-b]pyridin-3-amine;
19 N-(4-(trifluoromethylthio)benzyl)-6-trifluoromethyl-isoxazolo[5,4-b]pyridin-3-amine;
20 6-methyl-N-(4-(trifluoromethylthio)benzyl)-isoxazolo[5,4-b]pyridin-3-amine;
21 N-(1-(3,5-dimethyl-phenyl)ethyl)-7-trifluoromethyl-isoxazolo[5,4-c]pyridin-3-amine;
22 N-(1-(3,5-dimethylphenyl)ethyl)-6-methyl-isoxazolo[5,4-b]pyridin-3-amine;
23 N-(4-(trifluoromethylthio)benzyl)-7-trifluoromethyl-isoxazolo[5,4-c]pyridin-3-amine;
24 N-(4-(trifluoromethylthio)benzypisoxazolo[5,4-b]quinolin-3-amine;
25 4-chloro-N-(4-(trifluoromethylthio)benzyl)-isoxazolo[4,5-c]pyridin-3-amine;
26 4-pentylthio-N-(4-(trifluoromethylthio)-benzypisoxazolo[4,5-c]pyridin-3-amine;
27 N-(6-chloropyridin-3-yl)isoxazolo[5,4-b]pyridin-3-amine;
28 N-(6-chloropyridin-3-yl)-6-(trifluoromethypisoxazolo[5,4-b]pyridin-3-amine;
29 N-(6-chloropyridin-3-yDisoxazolo[5,4-c]pyridin-3-amine;
30 N-(6-chloropyridin-3-yl)-7-(trifluoromethypisoxazolo[5,4-c]pyridin-3-amine;
31 N-phenyl-6-(trifluoromethypisoxazolo[5,4-b]pyridin-3-amine;
32 N-phenyl-7-(trifluoromethyl)isoxazolo[5,4-c]pyridin-3-amine;
and the physiologically acceptable salts thereof.

9. A pharmaceutical composition comprising at least one isoxazolopyridine as claimed in claim 1,
in the form of an individual stereoisomer or a mixture thereof, in the form of a free compound and/or a physiologically acceptable salt thereof, and optionally one or more suitable additives and/or auxiliary substances and/or optionally further active ingredients.

10. A method of treating a disorder in a patient in need of such treatment, said disorder being at least one disorder selected from the group consisting of pain, epilepsy, urinary incontinence, anxiety, dependency, mania, bipolar disorders, migraine, cognitive diseases, and dystonia-associated dyskinesias, said method comprising administering to said patient an amount effective to treat said disorder of at least one isoxazolopyridine as claimed in claim 1, each in the form of an individual stereoisomer or a mixture thereof, in the form of a free compound and/or a physiologically acceptable salt thereof.

11. (canceled)
